(19) United States
(12) Patent Application Publication
(10) Pub. No.: US 2010/0209480 A1
(43) Pub. Date: Aug. 19, 2010

(54) GALENICAL FORMULATIONS OF ORGANIC COMPOUNDS

(76) Inventors: Ralf Altenburger, Eimeldingen (DE); Maggy B. Saunier, Jettingen (FR); Nicole Bargenda, Bad Laer (DE); Michael A. Bock, Freiburg (DE); Sabine Adler, Freiburg (DE); Bruno Buss, Osterholz-Scharmbeck (DE); Catherine Curdy, Riehen (CH); Indrajit Ghosh, Parsippany, NJ (US); Stefan Hirsch, Lorrach (DE); Patrice F. Keller, Muespach (FR); Charu Kochhar, Basel (CH); Shoufeng Li, Basking Ridge, NJ (US); Nicoletta Loggia, Basel (CH); Amol S. Matharu, Bedminster, NJ (US); Julien Taillemite, Huningue (FR); Wei-Qin Tong, Basking Ridge, NJ (US); Sudha Vippagunta, Morris Plains, NJ (US); Hong Wen, Westfield, NJ (US); Marie-Christine Wolf, St. Louis (FR); Jay P. Lakshman, Bridgewater, NJ (US); James Kowalski, Belle Mead, NJ (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/678,877

(22) PCT Filed: Sep. 24, 2008
(86) PCT No.: PCT/US08/77416

§ 371 Date: Mar. 18, 2010

Related U.S. Application Data
(60) Provisional application No. 60/975,919, filed on Sep. 28, 2007.
(60) Provisional application No. 60/975,909, filed on Sep. 28, 2007.
(60) Provisional application No. 60/975,901, filed on Sep. 28, 2007.
(60) Provisional application No. 60/975,925, filed on Sep. 28, 2007.

(51) Int. Cl. A61K 31/41; A61K 9/70; A61K 9/48; A61K 9/24; A61K 9/14; A61P 13/12; A61P 9/10; A61P 9/04; A61P 9/12; B29B 9/06

(52) U.S. Cl. 424/443; 424/451; 424/464; 424/484; 514/381; 264/140

(57) ABSTRACT

The present invention relates to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 80% or less after 10 minutes and 98% or less after 20 minutes, and a dissolution profile of component b) of 25% or more after 30 minutes, and 40% or more after 60 minutes at pH 4.5.

The present invention relates to pharmaceutical oral fixed dose combinations comprising an orally active renin inhibitor, Aliskiren, or a pharmaceutically acceptable salt thereof, and an angiotensin II antagonist, Valsartan, or a pharmaceutically acceptable salt thereof, as the active ingredients in a suitable carrier. In particular, the present invention provides galenical formulations comprising the hemi-fumarate salt of Aliskiren in combination with Valsartan. The present invention also relates to the processes for their preparation and to their use as medicaments.

Renin released from the kidneys cleaves angiotensinogen in the circulation to form the decapeptide angiotensin I. This is in turn cleaved by angiotensin converting enzyme in the lungs, kidneys and other organs to form the octapeptide angiotensin II. The octapeptide increases blood pressure both directly by arterial vasoconstriction and indirectly by liberating from the adrenal glands the sodium-ion-retaining hormone aldosterone, accompanied by an increase in extracellular fluid volume. Inhibitors of the enzymatic activity of renin bring about a reduction in the formation of angiotensin I. As a result a smaller amount of angiotensin II is produced. The reduced concentration of that active peptide hormone is the direct cause of, e.g., the antihypertensive effect of renin inhibitors. Accordingly, renin inhibitors, or salts thereof, may be employed, e.g., as antihypertensives or for treating congestive heart failure.

The renin inhibitor, Aliskiren, in particular, a hemi-fumarate thereof, is known to be effective in the treatment of reducing blood pressure irrespective of age, sex or race and is also well tolerated. Aliskiren in form of the free base is represented by the following formula

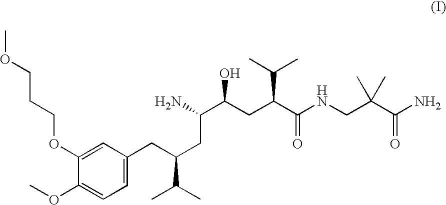

and chemically defined as 2(S),4(S),5(S),7(S)—N-(3-amino-2,2-dimethyl-3-oxopropyl)-2,7-di(1-methylethyl)-4-hydroxy-5-amino-8-[4-methoxy-3-(3-methoxy-propoxy)phenyl]-octanamide. As described above, most preferred is the hemi-fumarate salt thereof which is specifically disclosed in EP 678503 A as Example 83.

Valsartan is a known Angiotensin receptor blocker (ARB, angiotensin II antagonist) and the combination with Aliskiren is described, e.g. in WO02/40007.

Angiotensin II is a hormone that causes blood vessels to constrict. This, in turn, can result in high blood pressure and strain on the heart. It is known that angiotensin II interacts with specific receptors on the surface of target cells. Two receptor subtypes for angiotensin II, namely AT1 and AT2, have been identified thus far. In recent times, great efforts have been made to identify substances that bind to the AT1 receptor. Angiotensin receptor blockers (ARBs, angiotensin II antagonists) are now known to prevent angiotensin II from binding to its receptors in the walls of blood vessels, thereby resulting in lower blood pressure. Because of the inhibition of the AT1 receptor, such antagonists can be used, therefore, as anti-hypertensives or for the treatment of congestive heart failure, among other indications.

Administration of such pharmaceutical agents via the oral route is preferred to parenteral administration because it allow self-administration by patients whereas parenteral formulations have to be administered in most cases by a physician or paramedical personnel.

However, Aliskiren is a drug substance difficult to formulate due to its physicochemical properties and it is not trivial to make oral formulations in the form of tablets in a reliable and robust way. For example, Aliskiren has a needle shaped crystallization habit, which has a negative influence on the bulk properties of the drug substance, e.g., flow properties and bulk density. The compression behavior of the drug substance is poor, leading to weak interparticulate bonds and polymorphism changes under pressure. Aliskiren has a strong elastic component that also leads to weakening of interparticulate bonds. The drug substance quality is very variable with effect on the processability of a tablet, e.g., particle size distribution, bulk density, flowability, wetting behavior, surface area and sticking tendency. Moreover, Aliskiren is highly hygroscopic. After contact with water and removal of the water, the drug substance polymorphism changes to an amorphous state, which shows inferior stability compared to the crystalline state. In addition, in the particular case of high dose of Aliskiren or a pharmaceutically acceptable salt thereof (up to 300 mg of the free base per tablet) makes a high drug loading necessary in order to achieve a reasonable tablet size.

The combination of these hurdles makes a standard tablet manufacturing process extremely difficult. A solid oral dosage form of Aliskiren is described in WO2005/089729.

On the other hand, Valsartan has pH dependent solubility whereby it ranges from very slightly soluble in an acidic environment to soluble in a neutral environment of the gastrointestinal tract. Further, development of a patient-convenient oral dosage form of Valsartan is challenging due to its low bulk density.

Moreover, in general the development of oral fixed dose combination formulations using certain active ingredients is challenging. As used herein, “fixed dose combination” refers to a combination of defined doses of two drugs or active ingredients presented in a single dosage unit (e.g. a tablet or a capsule) and administered as such; further as used herein, “free dose combination” refers to a combination of two drugs or active ingredients administered simultaneously but as two distinct dosage units. When formulating oral fixed dose combinations, it is of advantage to provide a patient-convenient dosage form that is bioequivalent to the corresponding free dose combination of the same active ingredients in order to save time and costs in the development of the fixed dose combination. Development of fixed-dose combinations that are bioequivalent to the free dose combination is challenging due to the multiplicity of hurdles arising from pharmacokinetic and pharmaceutical properties of the drugs sought to be combined.

The difficulties encountered with Aliskiren to prepare oral formulations in the form of tablets in a reliable and robust way are believed to be potentiated when using it in combination with other therapeutic agents, in particular Valsartan for the reasons mentioned above.

In the case where the therapeutic doses of Valsartan and Aliskiren are high, when the two drugs are combined it is highly desired that the amounts of excipients are kept at a minimum to avoid excessively large formulations. Despite that fact, the formulation should still fulfill all of the above requirements.

Accordingly, a suitable and robust galenical formulation overcoming the above problems related to the properties of Aliskiren in particular when formulated together with Valsartan need to be developed.

Surprisingly it has been found that a certain dissolution profile of the two active ingredients is required in order to achieve a robust galenical formulation of the combination which is as similar as possible to the corresponding free dose combination with regard to the area under the curve (AUC) and preferably also the maximum plasma concentration (Cmax) so as to be most preferably bioequivalent to the free combination of the two active ingredients. From the solubility and absorption properties of the individual active ingredients one would not expect that the dissolution profile is critical in approaching or reaching bioequivalence.

In one embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 80% or less, preferably of 60% or less, more preferably of from 60% to 15%, after 10 minutes and 98% or less, preferably 95% or less, more preferably of from 95% to 40%, after 20 minutes, and a dissolution profile of component b) of 25% or more, preferably of 30% or more, after 30 minutes, and 40% or more, preferably 45% or more after 60 minutes at pH 4.5.

In a preferred embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 80% or less after 10 minutes and 98% or less after 20 minutes, and a dissolution profile of component b) of 25% or more after 30 minutes, and 40% or more after 60 minutes at pH 4.5; preferably an in vitro dissolution of component a) of 60% or less after 10 minutes and 95% or less after 20 minutes, and a dissolution profile of component b) of 25% or more after 30 minutes, and 45% or more after 60 minutes at pH 4.5; more preferably an in vitro dissolution of component a) of from 60% to 15%, after 10 minutes and of from 95% to 40%, after 20 minutes, and a dissolution profile of component b) of 30% or more, after 30 minutes, and 40% or more after 60 minutes at pH 4.5.

In a further embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 60% or less after 10 minutes and 95% or less after 20 minutes, and a dissolution profile of component b) of 25% or more after 30 minutes, and 45% or more after 60 minutes at pH 4.5.

In yet further embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of from 60% to 15%, after 10 minutes and of from 95% to 40%, after 20 minutes, and a dissolution profile of component b) of 30% or more, after 30 minutes, and 40% or more after 60 minutes at pH 4.5.

In another embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 60% or less after 10 minutes and 95% or less, preferably of from 95% to 40%, after 20 minutes, and a dissolution profile of component b) of 40% or less after 30 minutes, and 50% or less after 60 minutes at pH 1.

In yet another embodiment, the present invention is directed to a pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
   wherein the pharmaceutical oral fixed dose combination shows an in vitro dissolution of component a) of 50% or less after 10 minutes and 95% or less, preferably of from 95% to 30% after 20 minutes, and a dissolution profile of component b) of 75% or more after 30 minutes, and 85% or more after 60 minutes at pH 6.8.

Such a pharmaceutical oral fixed dose combination has an AUC and preferably also a Cmax for the respective active ingredients which is as similar as possible to the to a free dose combination of Aliskiren and Valsartan and such a pharmaceutical oral fixed dose combination is most preferably bioequivalent to such a free combination. It was surprising that the above dissolution data were so crucial since for Aliskiren and Valsartan it should not matter at which rate the active ingredient is released during the first 20 min and 60 min, respectively. As a BCS (biopharmaceutical classification system) class 3 compound (high solubility, low permeability), the release rate and subsequent dissolution rate for Aliskiren from the fixed dose combination should not be critical as long as the dissolution rate is similar or faster than for the existing Aliskiren film-coated tablets. Indeed, one of the pharmacokinetic parameters, the area under the curve (AUC) is taken over a period of 24 h so that the release rate and subsequent dissolution rate during the first 1 h or less should not be that important. Nevertheless, it was found that if the dissolution profile for at least one of the components, i.e. Aliskiren or Valsartan, typically the dissolution profile for Aliskiren, was outside the above-mentioned ranges, no similarity in AUC and/or Cmax and thus no bioequivalence for the fixed dose combination was found. For example in the case of Aliskiren, a faster dissolution than as mentioned above leads to a substantially lower exposure from the fixed combination compared to that from the free combination. It is surprising to find that an inverse relationship exists for Aliskiren between dissolution and absorption, whereby a dosage form with a faster dissolution of Aliskiren has lower bioavailability.

Throughout the present application, the various terms are as defined below:

Release profile:

The term “release” as used herein refers to a process by which the pharmaceutical oral fixed dose combination is brought into contact with a fluid and the fluid transports the drug(s) outside the dosage form into the fluid that surrounds the dosage form. The combination of delivery rate and delivery duration exhibited by a given dosage form in a patient can be described as its in vivo release profile. The release profiles of dosage forms may exhibit different rates and durations of release and may be continuous. Continuous release profiles include release profiles in which one or more active ingredients are released continuously, either at a constant or variable rate.

When two or more components that have different release profiles are combined in one dosage form, the resulting individual release profiles of the two components may be the same or different compared to a dosage form having only one of the components. Thus, the two components can affect each other's release profile leading to a different release profile for each individual component.

A two-component dosage form can exhibit release profiles of the two components that are identical or different to each other. The release profile of a two-component dosage form where each component has a different release profile may be described as “asynchronous”. Such a release profile encompasses both (1) different continuous releases where preferably component b) is released at a slower rate than component a), and (2) a profile where one of components a) and b), preferably component b), is released continuous and the other of components a) and b), preferably component a), is modified to be released continuous with a time delay. Also a combination of two release profiles for one drug is possible e.g. 50% of the drug in continuous and 50% of the same drug continuous with a time delay.

Immediate release:

For the purposes of the present application, an immediate release formulation is a formulation showing a release of the active substance(s), which is not deliberately modified by a special formulation design or manufacturing method.

Modified release:

For the purposes of the present application, a modified release formulation is a formulation showing a release of the active substance(s), which is deliberately modified by a special formulation design or manufacturing method. This modified release can be typically obtained by delaying the time of release of one or both of the components, preferably component a). Typically for the purposes of the present invention, a modified release refers to a release over 5 h, such as a release over 3 h or even shorter. Modified release as used herein is meant to encompass both a different continuous release over time of the two components or a delayed release where one of the components, preferably component a), is released only after a lag time. Such a modified release form may be produced by applying release-modifying coatings, e.g. a diffusion coating, to the drug substance(s) or to a core containing the drug substance(s), or by creating a release-modifying matrix embedding the drug substance(s).

The term “time delay” as used herein refers to the period of time between the administration of a dosage form comprising the composition of the invention and the release of the active ingredient from a particular component thereof.

The term “lag time” as used herein refers to the time between the release of the active ingredient from one component of the dosage form and the release of the active ingredient from another component of the dosage form.

Disintegration:

The term “disintegration” as used herein refers to a process where the pharmaceutical oral fixed dose combination, typically by means of a fluid, falls apart into separate particles and is dispersed. Disintegration is achieved when the solid oral dosage form is in a state in which any residue of the solid oral dosage form, except fragments of insoluble coating or capsule shell, if present, remaining on the screen of the test apparatus is a soft mass having no palpably firm core in accordance with USP <701>. The fluid for determining the disintegration property is water, such as tap water or deionized water. The disintegration time is measured by standard methods known to the person skilled in the art, see the harmonized procedure set forth in the pharmacopoeias USP <701> and EP 2.9.1 and JP.

Erosion:

The term “erosion” as used herein refers to a process by which the pharmaceutical oral fixed dose combination may be worn away, diminished or dissolved when placed in an external environment (e.g. dissolution medium, body fluids etc.). In contrast to disintegration, the pharmaceutical oral fixed dose combination is not dispersed by falling apart, rather it is becoming smaller with time as the erosion process proceeds.

Dissolution rate:

The term “dissolution” as used herein refers to a process by which a solid substance, here the active ingredients, is dispersed in molecular form in a medium. The dissolution rate of the active ingredients of the pharmaceutical oral fixed dose combination of the invention is defined by the amount of drug substance that goes in solution per unit time under standardized conditions of liquid/solid interface, temperature and solvent composition. The dissolution rate is measured by standard methods known to the person skilled in the art, see the harmonized procedure set forth in the pharmacopoeias USP <711> and EP 2.9.3 and JP. For the purposes of this invention, the test is for measuring the dissolution of the individual active ingredients is performed following pharmacopeia USP <711> at the pH as set forth herein for the different embodiments. In particular, at pH 4.5 and 1 the test is performed using a paddle stirring element at 75 rpm (rotations per minute), whereas at pH 6.8 the test is performed using a shaft stirring element at 100 rpm. At pH 4.5 or pH 6.8, the dissolution medium is preferably a buffer, typically a phosphate buffer, especially as described in the example “Dissolution Test”. The molarity of the buffer is preferably 0.1 M. At pH 6.8, the molarity of the buffer is preferably 0.05 M.

Physically separated:

The term “physically separated” as defined herein refers to a pharmaceutical oral fixed dose combination containing both components a) and b) formulated to minimize physical contact such that the dissolution profile is as similar as possible to the free dose combination of a) and b) with regard to the area under the curve (AUC) and preferably also the maximum plasma concentration (Cmax) so as to approach or reach bioequivalence. In one embodiment, “physically separated” refers to a pharmaceutical oral fixed dose combination containing both components a) and b) formulated such that they are not mixed with each other in the same carrier but are separated. This separation helps to minimize the interactions between the two components especially upon release of same. Typically the physical separation means that the two components a) and b) are present in different compartments, such as layers, or are present as different entities, such as particulates or granulates, of the formulation. It is not necessary that he two components a) and b) are further separated by additional layers or coating although this may be appropriate from case to case. This physical separation of the two components a) and b) in one dosage form can be achieved by various means known in the art.

In one embodiment, this is achieved by formulating the respective components a) and b) into separate layers, coats or shells, preferably layers or shells to obtain, e.g. a multi- or bilayer formulation, a dry-coated (core in a shell) tablet, a molded delivery system, or a spray coated tablet, preferably to obtain a bilayer formulation or a dry-coated formulation. Specific examples of such formulation techniques are described hereinafter.

In another embodiment, this is achieved by using particulate systems (multiparticulates) that comprise particles of different populations of component a) and component b), respectively, to obtain, e.g. capsules, sachets, stickpacks filled with multiparticulates, tablets obtained from compressing multiparticulates, and minitablets obtained from compressing multiparticulates, such as granules or beads, which can subsequently be filled into capsules. Another form of a physical separation is a capsule filled with 1) multiparticulates of one of the components and 2) one tablet, several tablets or minitablets obtained from compressing multiparticulates, such as granules or beads, of the other component.

One can also consider any combination of the above two approaches such as multiparticulates, such as pellets, or minitablets provided with a layer, coat or shell where the layer, coat or shell contains one of the components a) and b) and the multiparticulates or minitablets contain the other'of the components a) and b).

The term “particulate” as used herein refers to a state of matter which is characterized by the presence of discrete particles, pellets, beads or granules irrespective of their size, shape or morphology. When a plurality of particulates is present, these are referred to a multiparticulates. Typically, the particulates have an average size of lower than of from 3 mm, preferably of from 1 μm to 3 mm. By “average particle size” it is meant that at least 50% of the particulates have a particle size of less than about the given value, by weight. The particle size may be determined on the basis of the weight average particle size as measured by conventional particle size measuring techniques well known to those skilled in the art. Such techniques include, for example, sedimentation field flow fractionation, photon correlation spectroscopy, light scattering, and disk centrifugation. If a mixture of multiparticulates component a) and component b) are used, the multiparticulates of component a) and b) may be in the same form (e.g. granules) and/or size or the multiparticulate system for one of the components may be in one form (e.g. particles) and size and the multiparticulate system for the other component may be in a different form (e.g. granules) and/or size.

The term “small tablets” within the scope of this application denotes tablets with an overall size of from 3 to 5 mm.

The term “minitablets” within the scope of this application denotes small tablets with an overall weight of from 2 to 30 mg, e.g. of from 4 to 9 mg, such as 7 mg, in their uncoated form. Minitablets are a specific form of multiparticulates as defined herein. They can be prepared as described herein, including preparation from other, smaller multiparticulates, such as granules or beads. The minitablets may have any shape known to the skilled person for tablets, e.g. round e.g. with a diameter of from 1.25 to 3 mm; cylindrical e.g. having a convex upper face and convex lower face and e.g. with a cylindrical diameter and height independently of each other are of from 1 to 3 mm; or biconvex minitablets e.g. whose height and diameter are approximately equal and are of from 1.25 to 3 mm.

Preferably, multiparticulates have a modified release coating. Specifically, if a mixture of multiparticulates component a) and component b) are used, the respective multiparticulates comprise different modified release coatings in order to provide different modified release profiles.

As used herein the term “granulation excipient” refers to any pharmaceutically acceptable material or substance that can be melt-extruded or melt-granulated with a therapeutic compound as further described below. The granulation excipient, for example, can be a polymer or a non-polymeric material as further described below.

As used herein the term “polymer” refers to a polymer or mixture of polymers that have a glass transition temperature, softening temperature or melting temperature by itself or in combination both above or below melting point (or melting range) of the therapeutic compound. The glass transition temperature (“Tg”) is the temperature at which such polymer's characteristics change from that of highly viscous to that of relatively less viscous mass.

Bioequivalence:

The term “bioequivalence” as used herein is related to bioavailability as follows. The term “bioavailability”, as used herein, is defined as a measure of the rate and amount of active ingredient which reaches the systemic circulation unchanged following the administration of the dosage form. The bioavailability of pharmaceutical oral fixed dose combination of the present invention is compared with that of the corresponding free dose combinations. The test (fixed dose combination) and the reference (free dose combination) formulations are administered orally to the subjects, and plasma samples are collected over time. The plasma samples are analyzed for concentration of Valsartan and Aliskiren. The maximum plasma concentration (Cmax) and the area under the plasma concentration vs. time curve (AUC) are calculated. Log-transformed AUCO-tlast (AUC from time zero to the last measurable concentration sampling time), AUCO-∞ (AUC from time zero to infinity), Cmax of aliskiren and valsartan are analyzed separately using a linear mixed effects model, with fixed effects from sequence, treatment and period, and random effects from subject. A point estimate (ratio of geometric mean of Cmax or AUC for test versus reference formulation) and the corresponding 90% confidence intervals are used to evaluate bioequivalence. For the test and reference products to be bioequivalent, the 90% confidence intervals for both AUC and Cmax point estimates should fall within 0.8-1.25. Obtaining bioequivalence between test and reference products is challenging, particularly for combinations of active ingredients, and the result cannot be predicted a priori.

Whenever reference is made to an AUC being similar to the active ingredient in the free combination, it is meant that the AUC in the pharmaceutical oral fixed dose combination of the present invention has preferably a 90% confidence interval which should fall within 0.8-1.25 for the active ingredients.

Whenever reference is made to an Cmax being similar to the active ingredient in the free combination, it is meant that the Cmax in the pharmaceutical oral fixed dose combination of the present invention has preferably a 90% confidence interval which should fall within 0.8-1.25 for the active ingredients.

In a preferred embodiment, the pharmaceutical oral fixed dose combination of the present invention has a release profile for one or both of the active ingredients, in particular for Aliskiren, such that the AUC point estimate(s) are in the range of from 0.7 to 1.30, more preferably of from 0.75 to 1.25, most preferably of from 0.8 to 1.1.

In another embodiment, the pharmaceutical oral fixed dose combination of the present invention has a release profile for one or both of the active ingredients, in particular for Aliskiren, such that the 90% confidence interval for AUC(s) are, of from 0.65 to 1.35, more preferably of from 0.7 to 1.30, still more preferably of from 0.75 to 1.25, most preferably of from 0.8 to 1.25,

In another embodiment, the pharmaceutical oral fixed dose combination of the present invention has a release profile for one or both of the active ingredients, in particular for Aliskiren, such that the 90% confidence interval for Cmax(s) are, of from 0.4 to 1.35, more preferably of from 0.5 to 1.30, still more preferably of from 0.7 to 1.25, most preferably of from 0.8 to 1.25

It is preferred that at least the AUC(s), more preferably both the AUC(s) and the Cmax(s) are within the above-mentioned ranges.

By virtue of this the pharmaceutical oral fixed dose combination of the present invention will approach or preferably reach bioequivalence.

In a preferred embodiment of the present invention, component a) is present in an amount ranging of from 10 to 45%, such as 10 to 35%, by weight based on the total weight of the pharmaceutical oral fixed dose combination. These percentages refer to the hemifumarate salt of aliskiren, and if the free base or other salts are used, the percentages will be adapted accordingly.

In another preferred embodiment of the present invention component a) is present in an amount of from 12 to 45%, such as of from 12 to 40%, in one embodiment of from 12 to 30%, such as of from 12 to 25%, by weight based on the total weight of the pharmaceutical oral fixed dose combination. These percentages refer to the hemifumarate salt of aliskiren, and if the free base or other salts are used, the percentages will be adapted accordingly.

In yet another preferred embodiment, component a) is present in an amount of 20% or more, such as 25% or more, by weight based on the total weight of the oral dosage form. These percentages are based on the free base of component a) and if a salt is used the percentages will be adapted accordingly.

In still another preferred embodiment, component a) is present in an amount of from 15 to 35%, such as of from 20 to 30%, by weight based on the total weight of the oral dosage form. These percentages are based on the free base of component a) and if a salt is used the percentages will be adapted accordingly.

In a further preferred embodiment, component a) is present in an amount of 79% or more, such as 84% or more, by weight based on the total weight of the granules comprising component a). These percentages are based on the free base of component a) and if a salt is used the percentages will be adapted accordingly.

In yet a further preferred embodiment, component a) is present in an amount of from 70 to 95%, such as of from 75 to 90%, by weight based on the total weight of the granules comprising component a). These percentages are based on the free base of component a) and if a salt is used the percentages will be adapted accordingly.

It is preferred that component a) is present in an amount ranging of from 75 mg to 300 mg of the free base per unit pharmaceutical oral fixed dose combination.

In a preferred embodiment of the present invention, component a) is present in an amount ranging of from 75 to 300 mg, such as of from 75 to 150 mg, of the free base per unit pharmaceutical oral fixed dose combination, in particular 75, 150 or 300 mg, such as 150 or 300 mg.

In a preferred embodiment of the present invention, component b) is present in an amount ranging of from 8 to 45%, such as of from 10 to 30%, in particular of from 12 to 27%, by weight based on the total weight of the pharmaceutical oral fixed dose combination. These percentages are based on the free acid of component b) and if a salt is used, the percentages will be adapted accordingly.

In a preferred embodiment of the present invention, component b) is present in an amount of from 15 to 40%, such as of from 20 to 40%, such as of from 20 to 30%, by weight based on the total weight of the pharmaceutical oral fixed dose combination. These percentages are based on the free acid of component b) and if a salt is used, the percentages will be adapted accordingly.

In another preferred embodiment, component b) is present in an amount of 20% or more, such as 25% or more, such as 28% or more, by weight based on the total weight of the oral dosage form. These percentages are based on the free acid of component b) and if a salt is used the percentages will be adapted accordingly.

It is preferred that component b) is present in an amount ranging of from 75 to 350 mg, such as of from 100 to 200 mg, more preferably of from 80 mg to 320 mg, such as of from 160 to 320 mg, per unit dosage form, in particular 80, 160 or 320 mg, such as 160 or 320 mg, based on the free acid of b).

The weight ratio of component a) to component b) preferably ranges of from 1:0.001 to 1:5, more preferably of from 1:0.5 to 1:4 or 1:0.03 to 1:0.07. Most preferably, the weight ratio is of from 1:1.0 to 1.1; 1:2.1 to 2.2; or 1:0.005 to 0.006 based on the free base of a) and the free acid of b). Most preferably, components a) and b), are used in amounts of 75/80 mg, 75/160 mg, 150/80 mg, 150/160 mg, 300/320 mg, 300/160 mg or 150/320 mg, most preferably 150/160 mg, 300/320 mg, 300/160 mg or 150/320 mg of a)/b), based on the free base of a) and the free acid of b). In one embodiment it is preferred to use a high drug load using 300 mg of a) and/or 320 mg of b), most preferably 300/320 mg of a)/b). When using a salt, such as the hemifumarate for component a), the ratios will be adapted accordingly.

The terms “effective amount” or “therapeutically effective amount” refers to the amount of the active ingredient or agent which halts or reduces the progress of the condition being treated or which otherwise completely or partly cures or acts palliatively on the condition. The terms “drugs”, “active substances”, active ingredients”, “active agents” etc. as used herein refer to components a) and b) unless specified otherwise. Each of component a) or b) can be referred to as a “drug”, “active substance”, active ingredient”, “active agent” etc.

In the above and in the following the term “Aliskiren”, if not defined specifically, is to be understood both as the free base and as a salt thereof, especially a pharmaceutically acceptable salt thereof, such as a hemi-fumarate, hydrogen sulfate, orotate or nitrate, most preferably a hemi-fumarate thereof.

Aliskiren, or a pharmaceutically acceptable salt thereof, can, e.g., be prepared in a manner known per se, especially as described in EP 678503 A, e.g., in Example 83.

In the following the term “Valsartan”, if not defined specifically, is to be understood both as the free base and as a salt thereof, especially a pharmaceutically acceptable salt thereof, as described below.

Valsartan, or a pharmaceutically acceptable salt thereof, can, e.g., be prepared in a manner known per se. Preferred salts forms include acid addition salts. The compounds having at least one acid group (e.g., COOH or 5-tetrazolyl) can also form salts with bases. Suitable salts with bases are, e.g., metal salts, such as alkali metal or alkaline earth metal salts, e.g., sodium, potassium, calcium or magnesium salts, or salts with ammonia or an organic amine, such as morpholine, thiomorpholine, piperidine, pyrrolidine, a mono-, di- or tri-lower alkylamine, e.g., ethyl-, tert-butyl-, diethyl-, diisopropyl-, triethyl-, tributyl- or dimethylpropylamine, or a mono-, di- or trihydroxy lower alkylamine, e.g., mono-, di- or tri-ethanolamine. Corresponding internal salts may furthermore be formed. Salts which are unsuitable for pharmaceutical uses but which can be employed, e.g., for the isolation or purification of free compounds I or their pharmaceutically acceptable salts, are also included. Even more preferred salts are, e.g., selected from the mono-sodium salt in amorphous form; di-sodium salt of Valsartan in amorphous or crystalline form, especially in hydrate form, thereof. Mono-potassium salt of Valsartan in amorphous form; di-potassium salt of Valsartan in amorphous or crystalline form, especially in hydrate form, thereof. Calcium salt of Valsartan in crystalline form, especially in hydrate form, primarily the tetrahydrate thereof; magnesium salt of Valsartan in crystalline form, especially in hydrate form, primarily the hexahydrate thereof; calcium/magnesium mixed salt of Valsartan in crystalline form, especially in hydrate form; bis-diethylammonium salt of Valsartan in crystalline form, especially in hydrate form; bis-dipropylammonium salt of Valsartan in crystalline form, especially in hydrate form; bis-dibutylammonium salt of Valsartan in crystalline form, especially in hydrate form, primarily the hemihydrate thereof; mono-L-arginine salt of Valsartan in amorphous form; bis-L-arginine salt of Valsartan in amorphous form; mono-L-lysine salt of Valsartan in amorphous form; bis-L-lysine salt of Valsartan in amorphous form.

Most preferably, Valsartan is used as the free acid.

The pharmaceutical oral fixed dose combination according to the present invention needs to be selected appropriately to show the desired dissolution profile. Typically, the pharmaceutical oral fixed dose combination is a solid dosage form.

The pharmaceutical oral fixed dose combination of the present invention preferably exhibits release profiles of both components a) and b), more preferably component a) that are regarded as modified release profiles. The pharmaceutical oral fixed dose combination of the present invention preferably exhibits a release profile of component b) that is regarded as an immediate release profile. In a preferred embodiment of the present invention, the release profiles of the two components of the pharmaceutical oral fixed dose combination are asynchronous. In one embodiment, both components are released continuously with an asynchronous release profile, whereby one of the components, preferably component a), is modified to be released at a slower continuous rate. In another embodiment, one of the components, preferably component a), is released with a time delay so as result in a time lag of component a) compared to component b).

Preferably, the pharmaceutical oral fixed dose combination of the present invention is designed in such a way that components a) and b) are physically separated. Typical technologies and formulation principles for pharmaceutical oral fixed dose combinations capable to match the required dissolution profile according to the present invention include the formulation examples described below in more detail.

### Multilayer Tablets

In one embodiment, the present invention is in particular related to a pharmaceutical oral fixed dose combination in the form of a multilayer tablet. A multilayer tablet has at least two layers (bilayer tablet) or can have three, four, five or more layers. Each of the layers contains not more than one of the components. Preferably, the tablet has 2 layers with one of the components in one of the two layers, but it is also possible that in addition to these two layers the tablet contains further layers containing only carrier and which may function e.g. as separation layer(s) or outer coating layer(s). Alternatively, if more than two layers are present, the components may be present in more than one layer as long as they are not present together in the same layer. For practical purposes, a bilayer tablet is preferred but all information detailed below is equally applicable to multilayer tablets.

Multilayer tablets, in particular, bilayer tablets, according to the present invention are characterized in that one layer contains component a) and the other layer contains component b).

Multilayer tablets, in particular, bilayer tablets, can be manufactured by methods known in the art, in particular, the methods described for preparing the individual tablets containing either component a) or component b). Preferably, each of the layers can be prepared using wet or dry granulation. Examples for wet granulation are aqueous or organic wet granulation, in particular organic wet granulation as described below. Preferred examples of dry granulation include roller compaction as described e.g. below. Dry granulation methods are preferred since these circumvent the use of solvents and avoid additional drying steps. For the multilayer tablet, in particular, the bilayer tablet of the present invention, the individual layers can be prepared by the same or different processes for example one layer can be prepared by wet granulation and the second layer can be prepared by roller compaction or, most preferably, both layers can be prepared using roller compaction. In another preferred embodiment, the layer that contains component a) is prepared by roller compaction, wet granulation or melt extrusion, most preferably melt extrusion.

Pharmaceutically acceptable additives suitable for use in multilayer tablets, in particular bilayer tablets, according to the present invention include, without limitation, diluents or fillers, disintegrants, glidants, lubricants, binders, colorants and combinations thereof. Preferred pharmaceutically acceptable additives include fillers and binders. The amount of each additive in a pharmaceutical oral fixed dose combination may vary within ranges conventional in the art.

Suitable fillers include, without limitation, microcrystalline cellulose (e.g., cellulose MK GR), mannitol, sucrose or other sugars or sugar derivatives, Calcium hydrogen phosphate, low-substituted hydroxypropyl cellulose (L-HPC), hydroxyethyl cellulose, hydroxypropyl methyl cellulose, and combinations thereof, preferably, microcrystalline cellulose, e.g., products available under the registered trade marks AVICEL, FILTRAK, HEWETEN or PHARMACEL. When present, a filler in the layer containing component a) may be employed in an amount ranging of from 1% to 40%, preferably of from 10% to 30% by weight of the tablet (prior to any optional film coating). As regards the layer containing component b), when present, a filler may be employed in an amount ranging of from 1% to 40%, preferably of from 10% to 30% by weight of the tablet (prior to any optional film coating). Preferably, both layers contain a filler.

Suitable binders include, without limitation, polyvinylpyrrolidone (PVP), such as e.g., PVP K 30 or PVP90F, polyethylene glycols (PEG), e.g., PEG 4000, hydroxypropylmethyl cellulose, hydroxypropyl cellulose, both preferably of medium to high viscosity, e.g., viscosity grades 3 or 6 cps, pregelatinized starch and combinations thereof. A most preferred binder is PVP K 30 or PVP90F. It was found that the presence of binder in the layer containing component a) plays an important role in obtaining the desired dissolution profile. A roller compacted layer containing component a) preferably contains the binder in the internal phase and a wet-granulated layer containing component a) preferably contains the binder in the internal and in the external phase. When present, a binder in the layer containing component a) may be employed in an amount ranging of from 0.1% to 20%, preferably of from 0.5% to 15%, such as of from 0.7% to 10%, by weight of the multilayer, preferably bilayer, tablet (prior to any optional film coating). When present, a binder in the layer containing component b) may be employed in an amount ranging of from 0.1% to 20%, preferably of from 0.2% to 10% by weight of the multilayer, preferably bilayer tablet, (prior to any optional film coating). Preferably, the binder is omitted in the layer containing component b).

Suitable lubricants include, without limitation, magnesium stearate, aluminum or calcium silicate, stearic acid, cutina, PEG 4000-8000, talc and combinations thereof, preferably magnesium stearate. When present, a lubricant in the layer containing component a) may be employed in an amount ranging of from 0.1% to 5%, preferably of from 0.5% to 3%, by weight of the multilayer, preferably bilayer, tablet (prior to any optional film coating). When present, a lubricant in the layer containing component b) may be employed in an amount ranging of from 0.1% to 5%, preferably of from 0.5% to 3%, by weight of the multilayer, preferably bilayer tablet (prior to any optional film coating). Preferably, both layers contain a lubricant, in each case preferably both in the external and the internal phase.

Suitable disintegrants include, without limitation, carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP(CROSPOVIDONE), crosslinked CMC (Ac-Di-Sol), carboxymethylstarch-Na (PIRIMOJEL and EXPLOTAB). A most preferred disintegrant is crosslinked PVP, preferably PVPPXL. When present, a disintegrant in the layer containing component a) may be employed in an amount ranging of from 0.5% to 20%, preferably of from 1% to 3%, by weight of the multilater, preferably bilayer, tablet (prior to any optional film coating). When present, a disintegrant in the layer containing component b) may be employed in an amount ranging of from 1% to 20%, preferably of from 2% to 12%, by weight of the multilayer, preferably bilayer tablet (prior to any optional film coating). Preferably the disintegrant is absent in the layer containing component a), especially in a roller compacted layer containing component a). A wet granulated layer containing component a) may contain the disintegrant. Preferably the layer containing component b) includes a disintegrant.

Suitable glidants include, without limitation, colloidal silicon dioxide (e.g., Aerosil 200), magnesium trisilicate, powdered cellulose, starch, talc and combinations thereof. When present, a glidant in the layer containing component a) may be employed in an amount ranging of from 00.05% to 5%, preferably of from 0.1% to 1%, by weight of the multilayer, preferably bilayer, tablet (prior to any optional film coating). When present, a disintegrant in the layer containing component b) may be employed in an amount ranging of from 0.05% to 5%, preferably of from 0.1% to 1%, by weight of the multilayer, preferably bilayer tablet (prior to any optional film coating).

In a first embodiment, the pharmaceutical oral fixed dose combinations of invention are multilayer, preferably bilayer, tablet pharmaceutical oral fixed dose combinations of low friability. Preferably the friability is not more than 0.8%. The friability is measured by standard methods known to the person skilled in the art, see the harmonized procedure set forth in the pharmacopoeias USP <1216> and EP 2.9.7 and JP.

The pharmaceutical oral fixed dose combinations of the first embodiment of the invention are multilayer, preferably bilayer, tablet pharmaceutical oral fixed dose combinations of suitable hardness (e.g. an average hardness ranging of from 250 N to 300 N for bilayer forms). Such an average hardness is determined prior to the application of any film coating on the pharmaceutical oral fixed dose combinations. In that regard, a preferred embodiment of this invention is directed to pharmaceutical oral fixed dose combinations which are film-coated. Suitable film coatings are known and commercially available or can be made according to known methods. Typically the film coating material is a polymeric film coating material comprising materials such as hydroxypropylmethyl cellulose, polyethylene glycol, talc and colorant. Typically, a film coating material is applied in such an amount as to provide a film coating that ranges of from 1% to 6% by weight of the film-coated tablet.

A further embodiment of the present invention is a process for the manufacture of a multilayer, preferably a bilayer, tablet according to the present invention. For example, a bilayer tablet comprising one layer containing component a) and one layer containing component b) can be prepared by the following method, comprising the steps of (1) granulating component a) and pharmaceutically acceptable additives, optionally in the presence of a granulation liquid, to form an Aliskiren granulate; (2) granulating component b) and pharmaceutically acceptable additives to form a Valsartan granulate; (3) optionally drying resulting respective granulates; (4) sieving; (5) optionally mixing the respective granulates with outer phase excipients; and (6) compressing the Valsartan granulates and the Aliskiren granulates together to form a bilayer tablet. The details regarding the components a) and b) and pharmaceutically acceptable additives, i.e., source, amount, etc., are as set forth above.

Attention is drawn to the numerous known methods of granulating, drying sieving and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press. The blending steps can be accomplished using any suitable means. Typically the component, e.g. component a), and pharmaceutically acceptable additives are dispatched to a suitable vessel such as a diffusion blender or diffusion mixer. The drying step can be accomplished by using any suitable means. The sieving steps can be accomplished using any suitable means, e.g. using oscillating sieving. The screening step can be accomplished using any suitable means. The compacting step can be accomplished using any suitable means. Typically compacting is accomplished using a roller compactor with a compaction force ranging of from 20 kN to 60 kN, preferably 35 kN. Compaction may also be carried out by slugging the blended powders into large tablets that are then size-reduced. The milling step can be accomplished using any suitable means. Typically the compacted material is milled through a screening mill. Preferably the milled material is blended, often with a pharmaceutically acceptable additive such as a lubricant, in a diffusion blender

In the first step of the method, component a) is granulated with pharmaceutically acceptable additives, optionally in the presence of a granulation liquid, to form an Aliskiren granulate. The granulation liquid can be any liquid or liquid mixture well-known in the granulation art such as ethanol, a mixture of ethanol and water, a mixture of ethanol, water and isopropanol, said mixtures may contain a binder, such as those described herein. The process is then referred to as an organic wet granulation. A preferred mixture of ethanol and water ranges of from 50/50 to 99/1 (% w/w), most preferably it is 94/6 (% w/w). A preferred mixture of ethanol, water and isopropanol ranges of from 45/45/5 to 98/1/1 (% w/w/w), most preferably of from 88.5/5.5/6.0 to 91.5/4.5/4.0 (% w/w/w). In a preferred embodiment, the granulation is effected by an ethanolic solution of the binder and additional ethanol.

Aliskiren wet granulation is typically accomplished by using the following method (1) blending component a) and pharmaceutically acceptable additives in the presence of a granulation liquid to form a blended material; (2) drying the blended material, (3) sieving the blended material; and (4) screening the sieved material to isolate the adequate Aliskiren granulate fraction.

Alternatively, Aliskiren granulation is accomplished using another method (dry granulation) as follows: (1) blending component a) and pharmaceutically acceptable additives to form a blended material; (2) sieving the blended material; (3) blending the sieved material to form a final blend material; (4) compacting the final blend material to form a compacted material; (5) milling the compacted material to form a milled material; and (6) blending the milled material to form the Aliskiren granulate.

Particularly preferable is a roller compaction method whereby the step of compacting is performed using a roller compactor. In this case, the compacting step can be accomplished using any suitable means. Typically, compacting is accomplished by using a roller compactor with a compaction force (for development scale machines) ranging of from 2 kN to 6 kN i.O., preferably of from 3 to 5 kN. Compaction may also be carried out by slugging the blended powders into large tablets that are then size-reduced. Preferably, the device used is a Freund Corporation; Roller Compactor Type TF Mini. Using this equipment, the screw speed is suitably adjusted to ensure proper quality of the roller compacted material. Preferably, the screw speed is more than 15 rpm, such as 20 to 30 rpm. Moreover, using this equipment, the roll speed is suitably adjusted to ensure proper quality of the roller compacted material. Preferably, the roll speed is 3 to 5 rpm. It is also preferred that no pre-compression force is applied.

In another preferred embodiment component a) is granulated by a melt extrusion granulation method. It has been surprisingly found that multilayer, preferably bilayer, tablets according to the present invention wherein component a) is granulated by a melt extrusion granulation method are robust formulations. Such formulations show less variability in their dissolution profile and in other tablet characteristics such as hardness. Furthermore, the drug loading that can be achieved when component a) is granulated by a melt extrusion granulation method is higher than that achievable by either a wet granulation or a roller compaction method. Accordingly, the melt extrusion granulation provides, as a further benefit, means to reduce the tablet size, which can help improve patient compliance.

Aliskiren melt extrusion granulation is typically accomplished by using the following method:

(a) blending Aliskiren, or a pharmaceutical acceptable salt thereof, and optionally one or more granulation excipient, to give a preblended material;
(b) sieving the blended material to give a screened material;
(c) blending the sieved material to give a blended material;
(d) melt extruding the blended material to give an extrudate;
(c) cooling the extrudate to ambient temperature;
(d) milling the cooled extrudate;
(e) optionally blending the milled extrudate with one or more pharmaceutically acceptable excipients to give the final Aliskiren melt granulate.

In one embodiment step (d) takes place according to a method comprising the following steps, preferably by using a 50 mm extruder:
(d1) preheating of the extruder prior to feeding the material, preferably at a extrusion temperature such as; zones 1-3 of from 25° C. to 30° C., such as 25° C., zone-4 of from 50° C. to 80° C., such as 50° C., zone-5 of from 60° C. to 80° C., such as 60° C., zone-6 of from 70° C. to 100° C., such as 70° C., zones 7-8 of from 80° C. to 120° C., such as 80° C. and zones 9-10 of from 60° C. to 120° C., such as 60° C.,
(d2) running the extrusion process, preferably at a extrusion temperature such as; zones 1-3 of from 25° C. to 70° C., such as of from 25° C. to 35° C., such as 30° C., zones 4-6 of from 45° C. to 90° C., such as of from 45° C. to 55° C., such as 50° C., zones 7-8 of from 45° C. to 90° C., such as of from 45° C. to 55° C., such as 50° C. and zones 9-10 of from 40° C. to 120° C., such as of from 40° C. to 50° C. such as 45° C.

In another embodiment step (d) preferably takes place by using a 16 mm extruder, preferably running the extrusion process at an extrusion temperature such as; zone-1 of from 25° C. to 55° C., such as of from 25° C. to 30° C., such as 25° C., zone-2 of from 25° C. to 70° C., such as of from 25° C. to 30° C., such as 25° C., zone-3 of from 25° C. to 90° C., such as of from 25° C. to 30° C., such as 25° C., zone-4 of from 30° C. to 130° C., such as of from 30° C. to 50° C., such as 40° C. and zone-5 of from 50° C. to 130° C., such as of from 50° C. to 80° C., such as 70° C.

In another embodiment step (d) preferably takes place by using a 27 mm extruder, preferably running the extrusion process at an extrusion temperature such as; zones 1-3 of from 25° C. to 50° C., such as of from 25° C. to 35° C., such as 30° C., zone-4 of from 25° C. to 50° C., such as of from 25° C. to 40° C., such as 35° C., zone-5 of from 25° C. to 50° C., such as of from 25° C. to 40° C., such as 35° C., zone-6 of from 40° C. to 70° C., such as of from 40° C. to 50° C., such as 45° C. and zones 7-8 of from 40° C. to 70° C., such as of from 40° C. to 50° C., such as 45° C.

In a further embodiment, step (d) preferably takes place according to a method comprising the following steps, preferably by using a 50 mm extruder:
(d1) preheating of the extruder prior to feeding the material, at a extrusion temperature such as; zones 1-3 25° C., zone 4 50° C., zone-5 60° C., zone-6 70° C., zones 7-8 80° C. and zones 9-10 60° C.,
(d2) running the extrusion process, preferably at a extrusion temperature such as; zones 1-3 30° C., zones 4-6 50° C., zones 7-8 50° C. and zones 9-10 45° C.

In a still further embodiment, step (d) preferably takes place by using a 16 mm extruder, preferably running the extrusion process at an extrusion temperature such as; zone-125° C., zone-2 25° C., zone-3 25° C. and zone-4 40° C. and zone-5 70° C.

In a still further embodiment, step (d) preferably takes place by using a 27 mm extruder, preferably running the extrusion process at an extrusion temperature such as; zones 1-3 30° C., zone-4 35° C., zone-5 35° C., zone-6 45° C. and zones 7-8 of 45° C.

In a preferred embodiment, the melt extrusion operation utilizes a 50 mm, a 27 mm or a 16 mm extruder, preferably wherein the material is fed at a rate of from 1 to 80 Kg/h, preferably of from 1 to 60 Kg/h, such as 1 Kg/h, 9 Kg/h or 50 Kg/h.

In a preferred embodiment, alsikiren, or a pharmaceutical acceptable salt thereof, is melt granulated with one or more granulation excipient. In one embodiment, the granulation excipient is a polymer or mixture of polymers. Types of polymers include, but are not limited to, water-soluble, water-swellable, water insoluble polymers and combinations of the foregoing. Examples of polymers include, but are not limited to:
homopolymers and copolymers of N-vinyl lactams, e.g., homopolymers and copolymers of N-vinyl pyrrolidone (e.g., polyvinylpyrrolidone), copolymers of N-vinyl pyrrolidone and vinyl acetate or vinyl propionate;
cellulose esters and cellulose ethers (e.g., methylcellulose and ethylcellulose) hydroxyalkylcelluloses (e.g., hydroxypropylcellulose), hydroxyalkylalkylcelluloses (e.g., hydroxypropylmethylcellulose), cellulose phthalates (e.g., cellulose acetate phthalate and hydroxylpropylmethylcellulose phthalate) and cellulose succinates (e.g., hydroxypropylmethylcellulose succinate or hydroxypropylmethylcellulose acetate succinate);
high molecular polyalkylene oxides such as polyethylene oxide and polypropylene oxide and copolymers of ethylene oxide and propylene oxide (e.g. poly(propylene oxide) flanked by chains of poly(ethylene oxide), also known by the trade name pluronics);
polyacrylates and polymethacrylates (e.g., methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates));
polyacrylamides;
vinyl acetate polymers such as copolymers of vinyl acetate and crotonic acid, partially hydrolyzed polyvinyl acetate;
polyvinyl alcohol; and
oligo- and polysaccharides such as carrageenans, galactomannans and xanthan gum, or mixtures of one or more thereof.

In one embodiment the polymer is selected from the group consisting of polyalkylene oxides, polyvinylpyrrolidone, such as PVPK 30, cellulose polymers, such as hydroxypropylmethylcellulose (e.g. HPMC 3 cps) and hydroxypropyl cellulose (e.g. HPC-EXF) or mixtures thereof. Most preferably the polymer is hydroxypropyl cellulose (e.g. HPC-EXF). When present, the ratio of aliskiren to polymer is preferably of from 88:12 to 95:5, more preferably of from 88:12 to 94:7, most preferably is 93.25:6.75.

In another embodiment, the granulation excipient is a non-polymeric material. Examples of non-polymeric materials include, but are not limited, to esters, hydrogenated oils, oils, natural waxes, synthetic waxes, hydrocarbons, fatty alcohols, fatty acids, monoglycerides, diglycerides, triglycerides and mixtures thereof. In one embodiment the non-polymeric granulation excipient is a fatty acid, for example stearic acid.
Examples of esters, such as glyceryl esters include, but are not limited to, glyceryl monostearate, e.g., CAPMUL GMS from Abitec Corp. (Columbus, Ohio); glyceryl palmitostearate; acetylated glycerol monostearate; sorbitan monostearate, e.g., ARLACEL 60 from Uniqema (New Castle, Del.); and cetyl palmitate, e.g., CUTINA CP from Cognis Corp. (Düsseldorf, Germany), magnesium stearate and calcium stearate.
Examples of hydrogenated oils include, but are not limited to, hydrogenated castor oil; hydrogenated cottonseed oil; hydrogenated soybean oil; and hydrogenated palm oil. An example of oil includes sesame oil.
Examples of waxes include, but are not limited to, carnauba wax, beeswax and spermaceti wax. Examples of hydrocarbons include, but are not limited to, microcrystalline wax and paraffin. Examples of fatty alcohols, i.e., higher molecular weight nonvolatile alcohols that have of from 14 to 31 carbon atoms include, but are not limited to, cetyl alcohol, e.g., CRODACOL C-70 from Croda Corp. (Edison, N.J.); stearyl alcohol, e.g., CRODACOL S-95 from Croda Corp; lauryl alcohol; and myristyl alcohol. Examples of fatty acids which may have of from 10 to 22 carbon atoms include, but are not limited to, stearic acid, e.g., HYSTRENE 5016 from Crompton Corp. (Middlebury, Conn.); decanoic acid; palmitic acid; lauric acid; and myristic acid.

In a preferred embodiment, the melt extrusion granulation process is a continuous process. Said continuous process utilizes an equipment train that features various pieces of equipment for unit operations, such as mixing, sieving, granulating, milling, compressing, tableting or coating, linked together via transfer means, such as vacuum, gravity, convey belts, vibratory belts or bucket belts. The pharmaceutical materials (i.e., the raw materials, such as aliskiren or salt thereof, one or more pharmaceutically acceptable excipients or a mixture of the foregoing, intermediate drug products and final drug product) are continuously conveyed from one piece of unit operation equipment to the next piece of unit operation equipment without any intervention or assistance from a human operator of the equipment train. Therefore, the final result is a concatenation of a chain of independent unit operations into a single equipment train that allows for the feeding of raw materials into the equipment train upstream and having a solid oral dosage form, such as tablets, pills, caplets, capsules or sachets, preferably tablets, produced downstream.

An exemplary equipment train can comprise, for example, the following pieces: a blender; a extruder; a mill; and a tablet press. Any type of blender as known by one of ordinary skill may be used in the present invention, for example a bin blender. The extruder used in the present invention is configured for melt granulation. In general, a extruder includes a rotating screw(s) within a stationary barrel. Along the entire length of the screw, distributive kneading of the materials (e.g. aliskiren, or salt thereof, and optionally one or more granulation excipient) is provided by the rotation of the screw(s) within the barrel. The output of the extrude, extrudates, is transferred to a cooling tower. The cooling tower cools the extrudates to ambient temperature and once cooled, the extrudates may be transferred to an in-line mill for milling into granules. Preferably the extruder of the present invention is a twin-screw extruder, for example a 50 mm, 27 mm or a 16 mm twin-screw extruder. Any type of mill as known by one of ordinary skill may be used in the present invention, for example a Frewitt hammer mill using 2 mm screen with a rate of 2000 rpm. Any type of tablet press as known by one of ordinary skill in the art may also be used in the present invention. Examples of such tablet presses include, but are not limited to, low or high-speed presses, single/bi multilayer presses, and tablet-in-tablet presses. Tablet presses use forces between two and ninety kN to compress the milled materials.

In a preferred embodiment, the melt extrusion continuous process comprises, for example, the operations of extrusion, cooling, flaking and milling. Preferably the cooling operation utilizes a chiller flaker unit which cools the melted extrudate and cuts the formed solid sheets into small flakes. The flakes are conveyed into the mill, through a cooling tower, and are milled through a screen, for example a 2 mm screen.

In the second step of the method, component b) is granulated with pharmaceutically acceptable additives to form a Valsartan granulate. Valsartan granulation can be accomplished by any suitable means. In a preferred embodiment of this invention, Valsartan granulation is accomplished by (1) blending component b) and pharmaceutically acceptable additives to form a blended material; (2) sieving the blended material; (3) blending the sieved material to form a final blend material; (4) compacting the final blend material to form a compacted material; (5) milling the compacted material to get a milled material; and (6) blending the milled material to form the Valsartan granulate.

The blending of step (1 and 3) can be accomplished using any suitable means. Typically the component b) and pharmaceutically acceptable additives are dispatched to a suitable vessel such as a diffusion blender or diffusion mixer. The sieving of step (2) can be accomplished using any suitable means such as those described above. The compaction of step (4) can be accomplished using any suitable means. For example, typically for component b) compacting is accomplished using a roller compactor with a compaction force ranging of from 20 kN to 60 kN, preferably 35 kN. Compaction may also be carried out by slugging the blended powders into large tablets that are then size-reduced. The milling of step (5) can be accomplished using any suitable means. Typically the compacted material is milled through a screening mill. The blending of step (6) can be accomplished using any suitable means. Preferably the milled material is blended, often with a pharmaceutically acceptable additive such as a lubricant, in a diffusion blender.

In a further step of the method, pharmaceutically acceptable additives may be added to the valsartan granulates and/or the aliskiren granulates. This is described as adding additives in the outer phase. The respective Aliskiren and Valsartan granulates are referred to as the inner phase. The additives may be distributed partly in the granulate (in the inner phase) and partly in the outer phase, which is preferably the case in the described invention. Filler, lubricant and glidant (if present), more preferably lubricant, can be distributed partly in the inner and partly in the outer phase, binder (if present) is preferably only part of the inner phase.

In the final step of the method, the Valsartan granulate (including additives) and the Aliskiren granulates (including additives) are compressed together to form a bilayer tablet. Compression can be accomplished using any suitable means. Typically compression is accomplished by using a bilayer rotary tablet press. Typical compression force ranges of from 5 kN to 35 kN, preferably of from 12 kN to 45 kN. Preferably, the layer containing component b) is pre-compressed and the layer containing component a) is added to the resulting pre-compressed layer and then both layers are compressed.

Optionally, the method comprises the step of film coating the multilayer, preferably bilayer, tablet. The details regarding the film coating material, i.e., components, amounts, etc., are as described herein, Film coating can be accomplished using any suitable means, Suitable film coatings are known and commercially available or can be made according to known methods. Typically the film coating material is a polymeric film coating material comprising materials such as hydroxypropylmethyl cellulose, polyethylene glycol, talc and colorant. Typically, a film coating material is applied in such an amount as to provide a film coating that ranges of from 1% to 6% by weight of the film-coated tablet.

In one embodiment, in a multilayer tablet, according to the present invention, such as a bilayer tablet, component a) is present in an amount of 20% of more, such as 22% or more, such as 25% or more by weight based on the total weight of the pharmaceutical oral fixed dose combination. These percentages are based on the free base of component a) and if a salt is used the percentages will be adapted accordingly.

In another embodiment, in a multilayer tablet, according to the present invention, such as a bilayer tablet, component a) is present in an amount of 40% of more, such as 50% or more, such as 60%, by weight based on the total weight of the layer comprising component a). These percentages are based on the free base of component a) and if a salt is used the percentages will be adapted accordingly.

In a further embodiment, in a multilayer tablet, according to the present invention, such as a bilayer tablet, component a) is present in an amount of from 40 to 70%, such as 50 to 65%, such as 50 to 60%, by weight based on the total weight of the layer comprising component a). These percentages are based on the free base of component a) and if a salt is used the percentages will be adapted accordingly.

In a still further embodiment, in a multilayer tablet, according to the present invention, such as a bilayer tablet, component b) is present in an amount of 20% of more, such as 23% or more, such as 25% or more, such as 28% or more, by weight based on the total weight of the pharmaceutical oral fixed dose combination. These percentages are based on the free acid of component b) and if a salt is used the percentages will be adapted accordingly.

In yet another embodiment, in a multilayer tablet, according to the present invention, such as a bilayer tablet, component b) is present in an amount of 50% of more, by weight based on the total weight of the layer comprising component b). These percentages are based on the free acid of component b) and if a salt is used the percentages will be adapted accordingly.

In a still further embodiment, in a multilayer tablet, according to the present invention, such as a bilayer tablet, component b) is present in an amount of from 30% to 70% by weight based on the total weight of the layer comprising component b). These percentages are based on the free acid of component b) and if a salt is used the percentages will be adapted accordingly.

### Overencapsulated Tablets

In another embodiment, the present invention is in particular related to a pharmaceutical oral fixed dose, combination in the form of overencapsulated tablets.

Thus the present invention is in particular related to pharmaceutical oral fixed dose combination in the form of overencapsulated tablets. Overencapsulated tablets in accordance with the present invention are typically a capsule filled with 1) multiparticulates containing one of the components and 2) a tablet, tablets or minitablets obtained from compressing multiparticulates containing the other component. Preferably, component a) is in the form of a tablet, tablets or minitablets obtained from compressing multiparticulates and component b) is in the form of multiparticulates.

In general, tablets containing component a) or component b), preferably component a), are obtained from mixing the active ingredient with the respective additives and granulating the mixture before compression into a tablet. The exact methods for preparing the tablet as well as the type of additives that can be employed can be taken from WO2005/089729 for component a) and WO 97/49394, WO 00/38676 and WO 01/97805 for component b). One or more tablets prepared in such a manner can be used for overencapsulation depending on the desired dose.

Multiparticulates containing component a) or component b), preferably component b), that are filled into the capsule are typically in the form of granulates.

Multiparticulates can be manufactured by methods known in the art. Preferably, multiparticulates are granulates that can be prepared using wet or dry granulation. Examples for wet granulation are aqueous or organic wet granulation, in particular organic wet granulation as described below. Preferred examples of dry granulation include roller compaction as described e.g. below. Dry granulation methods are preferred since these circumvent the use of solvents and avoid additional drying steps. Most preferably, multiparticulates can be prepared by using roller compaction.

Multiparticulates may further contain excipients well known in the art. Pharmaceutically acceptable additives suitable for use in multiparticulates according to the present invention include, without limitation, diluents or fillers, disintegrants, glidants, lubricants, binders, surfactants, colorants and combinations thereof e.g. as described below. Preferred pharmaceutically acceptable additives include fillers, surfactants and binders. The amount of each additive in a pharmaceutical oral fixed dose combination may vary within ranges conventional in the art.

Suitable fillers include, without limitation, microcrystalline cellulose (e.g., cellulose MK GR), mannitol, sucrose or other sugars or sugar derivatives, Calcium hydrogen phosphate, low-substituted hydroxypropyl cellulose, hydroxyethyl cellulose, hydroxypropyl methyl cellulose, and combinations thereof, preferably, microcrystalline cellulose, e.g., products available under the registered trade marks AVICEL, FILTRAK, HEWETEN or PHARMACEL. When present, a filler may be employed in an amount ranging of from 1% to 40%, preferably of from 10% to 30% by weight of the multiparticulates.

Suitable binders include, without limitation, polyvinylpyrrolidone (PVP), such as e.g., PVP K 30 or PVP90F, polyethylene glycols (PEG), e.g., PEG 4000, hydroxypropylmethyl cellulose, hydroxypropyl cellulose, both preferably of medium to high viscosity, e.g., viscosity grades 3 or 6 cps, pregelatinized starch and combinations thereof. A most preferred binder is PVP K 30 or PVP90F. When present, a binder may be employed in an amount ranging of from 0.5% to 30%, preferably of from 1% to 20%, such as of from 2% to 15%, by weight of the multiparticulates.

Suitable lubricants include, without limitation, magnesium stearate, aluminum or calcium silicate, stearic acid, cutina, PEG 4000-8000, talc and combinations thereof, preferably magnesium stearate. When present, a lubricant may be employed in an amount ranging of from 0.1% to 5%, preferably of from 0.3% to 3%, by weight of the multiparticulates.

Suitable disintegrants include, without limitation, carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP(CROSPOVIDONE), crosslinked CMC (Ac-Di-Sol), carboxymethylstarch-Na (PIRIMOJEL and EXPLOTAB). A most preferred disintegrant is crosslinked PVP, preferably PVPPXL. When present, a disintegrant may be employed in an amount ranging of from 0.5% to 30%, preferably of from 1% to 20%, by weight of the multiparticulates.

Suitable surfactants include, without limitation, sodium laurylsulphate, sodium dodecyl sulfate cetomacrogol, a wax, glycerol monostearate, a sorbitan ester and a poloxamer, in particular Duponol C, and combinations thereof. When present, a surfactant in the layer containing component a) may be employed in an amount ranging of from 0.05% to 5%, preferably of from 0.1% to 1%, by weight of the multiparticulates.

Suitable glidants include, without limitation, colloidal silicon dioxide (e.g., Aerosil 200), magnesium trisilicate, powdered cellulose, starch, talc and combinations thereof. When present, a glidant may be employed in an amount ranging of from 0.05% to 5%, preferably of from 0.1% to 1%, by weight of the multiparticulates.

A preferred group of multiparticulates of component a) or component b) according to the invention are those having an effective average particle size of less than 1000 μm, preferably of from 10 to 800 μm, more preferably of from 30 to 500 μm. The drug microparticles consist of pure drug or may optionally be combined with one or more pharmaceutically acceptable excipients to form a drug excipient matrix, e.g. ethylcellulose or a methacrylic acid copolymer and a stabilizer, e.g. colloidal silica, to form the microparticle drug core, for instance by spray-drying, fluid-bed drying or precipitation techniques. Crystalline particles, e.g. in a size range of from 1 to 200 micron (μm), may also be prepared by means of high pressure homogenization of a suspension of unmilled crystalline drug crystals in any fluid in which the drug substance is sparsely soluble, such as organic solvents, e.g. cyclohexane.

These microparticulate drug suspensions may be directly coated with the aid of a polymer, or embedded in a polymer matrix, e.g. by adding the polymer and dissolving it in the homogenized suspension which is subsequently spray dried or spray layered. Preferably polymers used are Ethylcellulose or acrylic and methacrylic copolymers containing quaternary ammonium groups.

The precipitation techniques may also include the coacervation techniques, e.g. to separate a liquid phase of a coating material from a polymeric solution and wrapping of that phase as a uniform layer around suspended core particles. The resulting microparticles may be collected by filtration or centrifugation, washed with an appropriate solvent, and subsequently dried by standard techniques such as spray drying or fluidized bed drying.

These drug particles may then be further coated with modified release coating ingredients as disclosed herein, and optionally a stabilizer, e.g. colloidal silica. The modified release coating may be prepared for instance by fluid-bed coating and/or granulation or precipitation techniques. The proper coating technology can be selected by a person skilled in the art.

The resulting coated drug particles may optionally be combined with a diluent, e.g. as disclosed herein, for example lactose, mannitol or sucrose, a lubricant, e.g. as disclosed herein, for instance magnesium stearate.

In another embodiment component a) or component b) may optionally be combined with a binder or optionally with a diluent and a binder, e.g. as disclosed herein, and formed into granules, e.g. using a technique such as high or low shear granulation or fluid bed granulation to form the granule drug core. The granules obtained may then be coated with modified release coating ingredients. The granule drug core typically has a mean width of diameter of from 0.05 to 2 mm or preferably from 0.1 to 2 mm, or more preferably of from 0.15 to 1.5 mm. The amount of drug substance present in the core may be of from 1 to 95% or preferably of from 20 to 90%, or more preferably of from 50 to 90% by weight, based on the total weight of the granule drug core (i.e. excluding the coating).

Drug particles were the drug is in the form of crystals, amorphous particles or a mixture thereof can also be used for subsequent coating.

In another embodiment component a) or component b) may optionally be combined with one or more pharmaceutically acceptable extrusion aid(s), e.g. microcrystalline cellulose, an amylose pregelled starch, etc., binder(s), e.g. as herein disclosed, or diluents, e.g. as herein disclosed, and formed into pellets, e.g. using a technique such as extrusion spheronisation, direct pelletisation/high or low shear granulation, fluid bed granulation or spray drying/melt concealing to form the pellet drug core. The pellets obtained may be coated with modified release coating ingredients. The pellet drug core typically has a width of diameter of from 0.2 to 2 mm, preferably of from 0.5 to 1.4 mm. The amount of drug substance present in the core may be of from 1 to 95% by weight, based on the total weight of the pellet drug core (i.e. excluding the coating).

In another embodiment, the drug optionally in combination with a pharmaceutically acceptable binder, may be layered onto the surface of a pharmaceutically acceptable seed, typically a particle (e.g. a sphere) of sucrose, lactose, mannitol, starch, microcrystalline cellulose or any combination thereof, to form the bead drug core. Such layering may be solution layering or powder layering. Such a pharmaceutically acceptable seed is preferably a non-pareil sugar/starch sphere of 18-20 mesh, 25-30 mesh or 35-40 mesh, most preferably a non-pareil sugar starch sphere of 25-30 mesh or Cellets, i.e. microcrystalline cellulose beads e.g. from Pharmatrans Sanaq AG, in the size range of 100-1000 μm, more preferably of 100-200 and 200-355 μm. The beads obtained may be coated with modified release coating ingredients, e.g. as herein disclosed. The bead drug core typically has a width of diameter of from 0.2 to 2 mm, preferably of from 0.5 to 1.4 mm. The amount of drug substance present in the core may be of from 1 to 95% by weight, based on the total weight of the bead drug core (i.e. excluding the coating).

Multiparticulates in the form of e.g. beads or granulates can also be pressed into minitablets as known in the art and these minitablets are filled into the capsules.

Multiparticulates can be prepared preferably by granulation. If component a) is in the form of multiparticulates such as granulates, it can be prepared as follows. Component a) is granulated with pharmaceutically acceptable additives, optionally in the presence of a granulation liquid, to form an Aliskiren granulate. The granulation liquid can be any liquid or liquid mixture well-known in the granulation art such as ethanol, a mixture of ethanol and water, a mixture of ethanol, water and isopropanol, said mixtures may contain a binder, such as those described herein. The process is then referred to as an organic wet granulation. A preferred mixture of ethanol and water ranges of from 50/50 to 99/1 (% w/w), most preferably it is 94/6 (% w/w). A preferred mixture of ethanol, water and isopropanol ranges of from 45/45/5 to 98/111 (% w/w/w), most preferably of from 88.5/5.5/6.0 to 91.5/4.5/4.0 (% w/w/w). In a preferred embodiment, the granulation is effected by an ethanolic solution of the binder and additional ethanol, Aliskiren granulation can be accomplished by any suitable means. Aliskiren granulation is typically accomplished using the following method (wet granulation) (1) blending component a) and pharmaceutically acceptable additives in the presence of a granulation liquid to form a blended material; (2) drying the blended material, (3) sieving the blended material; and (4) screening the sieved material to isolate the adequate Aliskiren granulate fraction. Alternatively, Aliskiren granulation is accomplished using another method (dry granulation) as follows: (1) blending component a) and pharmaceutically acceptable additives to form a blended material; (2) sieving the blended material; (3) blending the sieved material to form a final blend material; (4) compacting the final blend material to form a compacted material; (5) milling the compacted material to form a milled material; and (6) blending the milled material to form the Aliskiren granulate.

Attention is drawn to the numerous known methods of granulating, drying sieving and mixing employed in the art, e.g., spray granulation in a fluidized bed, wet granulation in a high-shear mixer, melt granulation, drying in a fluidized-bed dryer, mixing in a free-fall or tumble blender, compressing into tablets on a single-punch or rotary tablet press. The blending steps can be accomplished using any suitable means. Typically the component a) and pharmaceutically acceptable additives are dispatched to a suitable vessel such as a diffusion blender or diffusion mixer. The drying of step can be accomplished using any suitable means, e.g. the sieving steps can be accomplished using any suitable means, e.g. using oscillating sieving. The screening step can be accomplished using any suitable means. The compacting step can be accomplished using any suitable means. Typically compacting is accomplished using a roller compactor with a compaction force ranging of from 20 kN to 60 kN, preferably 35 kN. Compaction may also be carried out by slugging the blended powders into large tablets that are then size-reduced. The milling step can be accomplished using any suitable means. Typically the compacted material is milled through a screening mill. Preferably the milled material is blended, often with a pharmaceutically acceptable additive such as a lubricant, in a diffusion blender.

If in a preferred embodiment, component b) is in the form of multiparticulates, such as granulates, it can be prepared as follows. Component b) is granulated with pharmaceutically acceptable additives to form a Valsartan granulate. Valsartan granulation can be accomplished by any suitable means. In a preferred embodiment of this invention, Valsartan granulation is accomplished by (1) blending component b) and pharmaceutically acceptable additives to form a blended material: (2) sieving the blended material: (3) blending the sieved material to form a final blend material; (4) compacting the final blend material to form a compacted material; (5) milling the compacted material to get a milled material; and (6) blending the milled material to form the Valsartan granulate.

The blending of step (1 and 3) can be accomplished using any suitable means. Typically the component b) and pharmaceutically acceptable additives are dispatched to a suitable vessel such as a diffusion blender or diffusion mixer. The sieving of step (2) can be accomplished using any suitable means such as those described above. The compaction of step (4) can be accomplished using any suitable means. For example, typically for component b) compacting is accomplished using a roller compactor with a compaction force ranging of from 20 kN to 60 kN, preferably 35 kN. Compaction may also be carried out by slugging the blended powders into large tablets that are then size-reduced. The milling of step (5) can be accomplished using any suitable means. Typically the compacted material is milled through a screening mill. The blending of step (a6) can be accomplished using any suitable means. Preferably the milled material is blended, often with a pharmaceutically acceptable additive such as a lubricant, in a diffusion blender.

The overencapsulated tablets can be prepared as follows:

1) the multiparticulates containing component a) or component b), preferably component b), are filled into a capsule and then one or multiple units of tablets containing component a) or component b), preferably component a), are subsequently added;
2) one or multiple units of tablets containing component a) or component b), preferably component a), are filled into a capsule and then multiparticulates containing component a) or component b), preferably component b), are subsequently added;
3) one unit of tablets containing component a) or component b), preferably component a), is filled into a capsule, multiparticulates containing component a) or component b), preferably component b), are subsequently added, and then one more unit of tablets containing component a) or component b), preferably component a), is added.

### Multilayered Molded Delivery Systems

In another embodiment, the present invention is in particular related to a pharmaceutical oral fixed dose combination in the form of multilayered molded delivery systems.

Thus, the present invention is related in particular to pharmaceutical oral fixed dose combination in the form of a molded multi-layered delivery system. This delivery system, in accordance with the present invention are typically multilayered delivery systems having two matrix zones, one containing component a) and the other containing component b). The matrix zones are designed to be erodible or disintegrable in an aqueous medium in which the composition is to be used. The matrix zones are preferably separated by a separation layer containing none of components a) or b). Optionally, pharmaceutical oral fixed dose combination has no separation layer. Preferably, the matrix zones and the optional separation layer are coated with a coating layer leaving at least one surface of each of the two matrix zones open to exposure by the aqueous medium. A preferred embodiment is as described in WO2006128471 where the matrix zone comprising component a) is placed on top of the matrix zone comprising component b), separated by a separation layer, and the formulation is further coated with a coating layer whereby the coating layer is left out on the top surface exposing the matrix zone comprising component a) and is left out on the bottom surface exposing the matrix zone comprising component b). The construction of the delivery system is preferably cylindrical or oval.

The matrix zones comprising either component a) or component b) may also contain other excipients as well, e.g. in order to improve the technical properties of the matrix zones so that it may be easier to produce or in order to improve the stability or obtain the desired release profile of the pharmaceutical oral fixed dose combination.

A suitable pharmaceutically acceptable excipient for use in the individual matrix zones may be selected from the group consisting of fillers, polymers, waxes, diluents, disintegrants, glidants, pH-adjusting agents, viscosity adjusting agents, solubility increasing or decreasing agents, osmotically active agents, stabilizers, surface active agents and solvents.

Suitable excipients for multilayered molded delivery systems include conventional tablet or capsule excipients. These excipients may be, for example, diluents such as dicalcium phosphate, calcium sulfate, lactose or sucrose or other disaccharides, cellulose, cellulose derivatives, kaolin, mannitol, dry starch, glucose or other monosaccharides, dextrin or other polysaccharides, sorbitol, inositol or mixtures thereof; binders such as acacia, sodium alginate, starch, gelatin, saccharides (including glucose, sucrose, dextrose and lactose), molasses, extract of Irish moss, panwar gum, ghatti gum, mucilage of isapol husk, carboxymethylcellulose, methylcellulose, veegum, larch arabolactan, polyethylene glycols, ethylcellulose, water, alcohols, waxes, polyvinylpyrrolidone such as, e.g., PVP K90 (may be used to improve mixing of the polymer with the other ingredients) or mixtures thereof; lubricants such as talc, magnesium stearate, calcium stearate, staeric acid, hydrogenated vegetable oils, sodium benzoate, sodium chloride, leucine, carbowax 4000, magnesium lauryl sulfate, colloidal silicon dioxide and mixtures thereof, disintegrants such as starches, clays, cellulose derivatives including crosscarmellose, gums, aligns, various combinations of hydrogencarbonates with weak acids (e.g. sodium hydrogencarbonate/tartaric acid or citric acid) crosprovidone, sodium starch glycolate, agar, cation exchange resins, citrus pulp, veegum HV, natural sponge, bentonite or mixtures thereof; volatile solvents such as alcohols, including aqueous alcohols, petroleum benzine, acetone, ether or mixtures thereof; plasticizers such as sorbitol and glycerine; and others such as cocoa butter, polyethylene glycols or polyethylene oxides, e.g. with a molecular weight of from 1,000-500,000 daltons, typically of from 1,000-100,000 daltons, more typically of from 1,000-50,000 daltons, especially of from 1,000-10,000 daltons, in particular of from 1,500-5,000 daltons, and mixtures thereof, hydrogenated vegetable oils, glycerinated gelatin or mixtures thereof.

Examples of suitable fillers are also dextrin, sucralfate, calcium hydroxyl-apatite and calcium phosphates.

The filler may be added in an amount so that the combination of the filler and the active substance, i.e. either component a) or component b) comprises up to 60%, typically up to 50%, by weight of the first composition.

In order to soften the carrier system, a plasticziser may be incorporated in the matrix zones. A suitable plasticizer is selected from the group consisting of phosphate esters; phthalate esters; amides; mineral oils; fatty acids and esters; fatty alcohols, vegetable oils and hydrogenated vegetable oils including acetylated hydrogenated cottonseed glyceride and acetylated hydrogenated soybean oil glycerides; acetyl tributyl citrate, acetyl triethyl citrate, Castor oil, diacetylated monoglycerides, dipropylene glycol salicylate glycerin, glyceryl cocoate, mono- and di-acetylated monoglycerides, nitrobenzene, carbon disulfide, β-naphtyl salicylate, phthalyl glycolate, diocyl phthalate; sorbitol, sorbitol glyceryl tricitrate; sucrose octaacetate; a-tocopheryl polyethylene glycol succinate, phosphate esters; phthalate esters; amides; mineral oils; fatty acids and esters; fatty alcohols; and vegetable oils, fatty alcohols including cetostearyl alcohol, cetyl alcohol, stearyl alcohol, oleyl alcohol and myristyl alcohol; methyl abietate, acetyl tributyl citrate, acetyl triethyl citrate, diisooctyl adipate, amyl oleate, butyl ricinoleate, benzyl benzoate, butyl and glycol esters of fatty acids, butyl diglycol carbonate, butyl oleate, butyl stearate, di(beta-methoxyethyl)adipate, dibutyl sebacate, dibutyl tartrate, diisobutyl adipate, dihexyl adipate, triethylene glycol di(beta-ethyl butyrate), polyethylene glycol di(2-ethyl hexoate), diethylene glycol monolaurate, monomeric polyethylene ester, hydrogenated methyl ester of rosin, methoxyethyl oleate, butoxyethyl stearate, butyl phthalyl butyl glycolate, glycerol tributyrate, triethylene glycol dipelargonate, beta-(p-tert-amyl phenoxy)ethanol, beta(p-tert-butylphenoxy)ethanol, beta-(p-teft-butylphenoxyethyl)acetate, bis(beta-p-tert-buthylphenoxydiethyl)ether, camphor, Cumar W-1, Cumar MH-1, Cumar V-1, diamyl phthalate, (diamylphenoxy)ethanol, diphenyl oxide, technical hydroabietyl alcohol, beckolin, benzene hexahydrochlonde, Clorafin 40, A-5, Piccalastic A-25, Flexol B400, Glycerol alfa-methyl alfa-phenyl ether, chlorinated naphthalene, HB-40, monoamylphthalate, Nevillac 10 o-nitrodiphenyl and Paracril 26.

The separation layer, if present, contains none of components a) or b) and may contain suitable excipients as listed above for the matrix zones.

In a preferred embodiment, the delivery system of the invention further comprises a coating layer having at least one opening exposing at least one surface of each of the two matrix zones, the coating being one which crumbles and/or erodes upon exposure to the aqueous medium at a rate which is equal to or slower than the rate at which the matrix zones erode in the aqueous medium, allowing exposure of said surfaces of the matrix zones to the aqueous medium to be modified. Coatings of this type are described in WO 95/22962, to which reference is made. These coatings comprise:
(a) a first cellulose derivative which has thermoplastic properties and which is substantially insoluble in the aqueous medium in which the composition is to be used, e.g. an ethylcellulose such as ethylcellulose having an ethoxyl content in the range of from 44.5-52.5%, or cellulose acetate, cellulose propionate or cellulose nitrate; and at least one of:
(b) a second cellulose derivative which is soluble or dispersible in water, e.g. a cellulose derivative selected from the group consisting of methylcellulose, carboxymethyl cellulose and salts thereof, cellulose acetate phthalate, microcrystalline cellulose, ethylhydroxyethylcellulose, ethylmethylcellulose, hydroxyethylcellulose, hydroxyethylmethylcellulose, hydroxypropylcellulose, hydroxypropylmethylcellulose, hydroxymethylcellulose and hydroxymethylpropylcellulose;
(c) a plasticizer, e.g. selected from the group consisting of phosphate esters; phthalate esters; amides; mineral oils; fatty acids and esters thereof with polyethylene glycol, glycerin or sugars; fatty alcohols and ethers thereof with polyethylene glycol, glycerin or sugars; and vegetable oils; or a non-ionic surfactant; and
(d) a filler, e.g. selected from conventional tablet or capsule excipients such as diluents, binders, lubricants and disintegrants.

The content of components a) and b) in the individual matrix zones may vary within wide limits. Typically, they may each suitably be present in an amount of up to 60%, typically up to 50%, by weight of the respective matrix zone.

The systems may be produced by methods known per se in the art, e.g. using methods described in WO 89/09066. WO 91/04015 and WO 95/22962, or using other methods known either in the pharmaceutical industry or used in the production of polymer-based materials. One important advantage of the compositions of the invention is that they may be produced by relatively simple and inexpensive methods. For compositions without a coating, any suitable extrusion or injection moulding method and apparatus may be used. For compositions provided with a coating, non-limiting examples of suitable production methods include the following co-extrusion and injection moulding techniques known in the art, such as injection moulding of the coating layer and subsequent injection moulding of the two matrix layers and injection moulding of the two matrix containing into a pre-formed tube which forms the coating.

Most preferably the delivery system is fabricated using injection molds as described e.g. in WO2006128471. For production of an inner separation layer, a mould is used. The mould is cooled and is placed on a glass plate and the molten/softened inner separation layer composition is poured on the mould and, if necessary, pressed into the holes of the mould. The inner plugs are weighted to ensure the uniformity of mass before use. The matrix is coated with a polymeric material (e.g. composition containing Ethylcellulose) using the procedure described in EP 0493 513 BI. The inner and outer plugs are assembled by pressing the cold inner plug into the coat with a cold metal pin. The outer plugs are assembled in the same manner. The finished coated composition is stored in the refrigerator until use. Automated machines for the purpose are commercially available, as this technology originated from the plastic industry. This methodology is used for manually making the delivery system.

### Dry-Coated Tablets

In another embodiment, the present invention is in particular related to a pharmaceutical oral fixed dose combination in the form of dry-coated tablets.

Thus the present invention is related in particular to a pharmaceutical oral fixed dose combination in the form of a tablet having a core containing an active agent and a compression coating (‘dry coat’, ‘dry coating layer’ or ‘shell’) surrounding the core, said compression coating containing also an active agent. This pharmaceutical oral fixed dose combination is referred to in the art as a press-coated, dry-coated or core-in-a-shell-tablet. Either component a) or component b) could be present in the core of the tablet and the shell of the tablet, respectively. Preferably, component a) is present in the core and component b) is present in the shell.

In one embodiment, the pharmaceutical oral fixed dose combination in the form of a dry-coated tablet comprises a core comprising component a), said core being surrounded by a shell comprising component b), wherein said pharmaceutical oral fixed dose combination shows an in vitro dissolution of component a) of 60% or less after 10 minutes and 95% or less after 20 minutes, and a dissolution profile of component b) of 25% or more after 30 minutes, and 45% or more after 60 minutes at pH 4.5; more preferably an in vitro dissolution of component a) of from 60% to 15%, after 10 minutes and of from 95% to 40%, after 20 minutes, and a dissolution profile of component b) of 30% or more, after 30 minutes, and 40% or more after 60 minutes at pH 4.5.

In another embodiment, the pharmaceutical oral fixed dose combination in the form of a dry-coated tablet comprises a core comprising component b), said core being surrounded by a shell comprising component a), wherein said pharmaceutical oral fixed dose combination shows an in vitro dissolution of component a) of 80% or less after 10 minutes and 98% or less after 20 minutes, and a dissolution profile of component b) of 25% or more after 30 minutes, and 40% or more after 60 minutes at pH 4.5; more preferably an in vitro dissolution of component a) of 60% or less after 10 minutes and 95% or less after 20 minutes, and a dissolution profile of component b) of 25% or more after 30 minutes, and 45% or more after 60 minutes at pH 4.5.

Dry-coated tablets are also particularly useful to administer active substances in a time-controlled manner. Preferably, the core is reliably and accurately positioned within the shell to obtain tablets having precisely defined release profiles. This is particularly advantageous when the tablet is adapted to release component a) from the core only after a defined lag time following dissolution of the shell.

In a preferred embodiment, a core may contain of from 0.1 to 90% by weight, more particularly of from 1 to 70%, still more particularly of from 1 to 50% by weight of component a) based on the total weight of the core. Typically the dry coat (shell) may contain of from 0.1 to 90% by weight, more particularly of from 1 to 70%, still more particularly of from 1 to 50% by weight of component b) based on the total weight of the dry coat (shell) of the dosage form.

In another embodiment, a core may contain of from 0.1 to 90% by weight, more particularly of from 1 to 70%, still more particularly of from 1 to 50% by weight of component b) based on the total weight of the core. Typically the dry coat (shell) may contain of from 0.1 to 90% by weight, more particularly of from 1 to 70%, still more particularly of from 1 to 50% by weight of component a) based on the total weight of the dry coat (shell) of the dosage form.

The core as well as the shell can be manufactured using methods known in the art such as e.g, direct blending, wet or dry granulation.

Similarly, any conventional tabletting additives like fillers, binders, lubricants and others may be employed in both core and shell of the dosage form. The particular core and/or shell excipients according to the present invention include, without limitation, diluents or fillers, disintegrants, glidants, lubricants, binders, colorants and combinations thereof. Preferred pharmaceutically acceptable additives include fillers and binders. The amount of each additive in a pharmaceutical oral fixed dose combination may vary within ranges conventional in the art. In a preferred embodiment, the core contains component a) as well as common tabletting excipients such as binders, fillers, disintegrants, lubricants and others as described herein. The shell preferably contains component b) as well as common tabletting excipients such as binders, fillers, disintegrants, lubricants and others as described herein.

Suitable fillers include, without limitation, microcrystalline cellulose (e.g., cellulose MK GR), mannitol, sucrose or other sugars or sugar derivatives, Calcium hydrogen phosphate, low-substituted hydroxypropyl cellulose, hydroxyethyl cellulose, hydroxypropyl methyl cellulose, and combinations thereof, preferably, microcrystalline cellulose, e.g., products available under the registered trade marks AVICEL, FILTRAK, HEWETEN or PHARMACEL. When present, a filler in each of the core and/or shell may be employed in an amount ranging of from 1% to 40%, preferably of from 10% to 30% by weight of the tablet (prior to any optional film coating).

Suitable binders include, without limitation, polyvinylpyrrolidone (PVP), such as e.g., PVP K 30 or PVP90F, polyethylene glycols (PEG), e.g., PEG 4000, hydroxypropylmethyl cellulose, hydroxypropyl cellulose, both preferably of medium to high viscosity, e.g., viscosity grades 3 or 6 cps, pregelatinized starch and combinations thereof. When present, a binder in each of the core and/or shell may be employed in an amount ranging of from 0.1% to 20%, preferably of from 0.5% to 15%, such as 0.7% to 10%, by weight of the tablet (prior to any optional film coating).

Suitable lubricants include, without limitation, magnesium stearate, aluminum or calcium silicate, stearic acid, cutina, PEG 4000-8000, talc and combinations thereof, preferably magnesium stearate. When present, a lubricant in each of the core and/or shell may be employed in an amount ranging of from 0.1% to 5%, preferably of from 0.5% to 3%, by weight of the tablet (prior to any optional film coating).

In one embodiment, for the core as well as the shell tablet materials may be chosen to provide an immediate release effect upon contact with moisture, liquids or fluids and as such may contain any of the known disintegrating or effervescing excipients to achieve this purpose. Alternatively, the skilled person may wish to have a modified release of component a) or b), preferably component a), and therefore employ excipients, or mixtures of excipients that form a gel matrix or eroding system when contacted with physiological or other media, thereby to permit of slow diffusion of the component a) or b), preferably component a), in a modified release manner.

These agents useful in the exercise of the present invention may be materials that effervesce in the presence of aqueous media thereby to provide the force necessary to mechanically disrupt the core and/or shell or cause erosion of the core and/or shell of the dosage form. Standard disintegrants used in the manufacturing of solid dosage forms do not or do not substantially swell or gel and prevent release of the active agent. Suitable disintegrants include, without limitation, carboxymethylcellulose calcium (CMC-Ca), carboxymethylcellulose sodium (CMC-Na), crosslinked PVP (e.g. CROSPOVIDONE, POLYPLASDONE or KOLLIDON XL), alginic acid, sodium alginate and guar gum, most preferably crosslinked PVP(CROSPOVIDONE), crosslinked CMC (Ac-Di-Sol), carboxymethylstarch-Na (PIRIMOJEL and EXPLOTAB). Preferably the core of the dosage form as well as the shell might contain, in addition to the active agent, cross-linked polyvinyl pyrollidone and/or croscarmellose sodium. When present, a disintegrant in each of the core and/or shell may be employed in an amount ranging of from 0.5% to 20%, preferably of from 1% to 10%, more preferably of from 1% to 3%, by weight of the tablet (prior to any optional film coating). In particular, cross-linked polyvinyl pyrolidone (e.g. CROSPOVIDONE, POLYPLASDONE or KOLLIDON XL) may be employed in the core and shell, respectively, in the amounts disclosed in relation to the core and shell, respectively. The polyvinyl pyrolidone is preferably present in amounts of from 1 to 25% by weight of the core or shell, more particularly of from 4 to 12%. Croscarmellose sodium is an internally cross-linked sodium carboxymethyl cellulose (also known as Ac-Di-Sol). It may be used in amounts of from 5 to 30% by weight based on the core, preferably of from 10 to 25%, e.g. of from 15 to 20% by weight. Any other common agent which has disintegrating or eroding effects on the core and/or shell such as e.g. pre-gelatinized starch could also be part of the core and shell formulation.

Suitable glidants include, without limitation, colloidal silicon dioxide (e.g., Aerosil 200), magnesium trisilicate, powdered cellulose, starch, talc and combinations thereof. When present, a glidant i in each of the core and/or shell may be employed in an amount ranging of from 00.05% to 5%, preferably of from 0.1% to 1%, by weight of the tablet (prior to any optional film coating).

The core and/or shell may as well contain hydrophilic or hydrophobic excipients to either increase or decrease the rate of ingress of media with regards to the core of the dosage form. Other core and/or shell materials may be employed that disisntegrate upon contact with physiological environments of certain pH values, or in response to the action of physiological reactive media such as enzymes. Other core and/or shell materials may be employed that simply provided an aesthetic aspect such as pleasant tasting material excipients. Any conventional tabletting excipients may be employed as well in the shell to achieve mechanical stability of the dosage form.

Excipients also used could be calcium phosphate salts such as dibasic calcium phosphate dihydrate, and may be present in an amount of from 5 to 90%, preferably of from 10 to 90% by weight of the core or shell, preferably of from 10 to 80%, more preferably of from 20 to 80% e.g. of from 10 to 45% or 40 to 75%.

The core as well as the shell formulation may in addition contain other common tablet excipients such as colorants, diluents, and taste-masking agents or flavourants.

Examples of excipients include colorants such a ferric oxide, e.g. yellow ferric oxide; lubricants such as magnesium stearate, stearic acid, cutina HR (hydrogenated ricinus oil); and glidants such as silicon dioxide, e.g. colloidal silicon dioxide. Ferric oxides may be used in amounts of from 0.01 to 0.5% by weight based on the core or shell, respectively; magnesium stearate may be present in amounts of 1 to 20% by weight of the core or shell, respectively; and colloidal silica may be used in amounts of from 0.1 to 20% by weight of the core or shell, respectively.

The core of the dosage form as well as the final dosage form may be a coated or non-coated dosage form. In case of a coated dosage form, a non-functional coat or functional coat, more preferably a non-functional coat, using conventional coating excipients such as e.g. Methylcellulose, Hydroxypropylcelluslose, Hydroxypropylmethylcellulose, Hydroxyethylcellulose, Polyethyleneglycol and—derivates, Polyvinylalcohol and—derivates, Talcum. Titanium oxide and iron oxides could be applied.

Another embodiment of the present invention is a process for the manufacture of dry-coated tablets, according to the present invention. The tablets may be formed on conventional press coating equipment. For example, the following process may be employed. A series of dies are arranged on a rotating platform. The dies are removably mounted in the platform such that differently sized dies may be employed as appropriate. Each die is hollow to receive a lower punch. The punch is positioned within the die such that the upper surface of the punch and the inner surface of the die define a volume for receiving a precise amount of shell material. Once loaded, die is rotated on the platform until it is positioned under an upper punch. The upper punch is then urged down onto the core material and the core material is pre-compressed or tamped between the upper and lower punch. A pre-formed core is then fed into the die to rest accurately in the required position on the tamped coating. Conventional press coating apparatus are equipped with centering devices that enable cores to be positioned—both vertically and radially—accurately in the required position to guarantee mechanical stability of the final dosage form. This might be achieved e.g. by a tamping process, whereby an initial amount of coating material is placed in a die and is tamped with a shaped punch that leaves an indentation in the coating material in which to receive a core. Thereafter, in a second filling operation, a precise amount of shell material is fed into the die to cover the core, and an upper punch compresses the coating material shell to form tablets according to the present invention.

The compression force applied during the tamping process is commonly low and is just sufficient to provide a bed for the core and to prevent movement of the coating material as a result of centrifugal force. Subsequent compression to form the coated tablet may be adjusted to give tablets of requisite hardness. Preferably, this compression force is high enough to guarantee mechanical stability of the dosage form but limited in order not to damage the punches.

The amount of shell material fed into the die can be precisely defined taking into consideration to the density of the shell material to ensure, after compression that the tablet is formed with the required shell thickness. Should it be necessary to change the thickness of the shell, die of appropriate internal dimensions may be placed in the rotating platform, and the amount of shell material fed into the die may be changed accordingly.

Suitable rotary tablet machines having high process speeds are known in the art and need no further discussion here.

The hardness of the tablet is preferably as high as required to achieve desired dissolution profile and mechanical stability of the dosage form for further processing. Hardness may be measured according to a process described in The European Pharmacopoeia 4, 2.9.8 on page 201. The test employs apparatus consisting of 2 opposing jaws, one of which moves towards the other. The flat surfaces of the jaws are perpendicular to the direction of movement. The crushing surfaces of the jaws are flat and larger than the zone of contact with the tablet. The apparatus is calibrated using a system with a precision of one Newton. The tablet is placed between the jaws. For each measurement, the tablet is oriented in the same way with respect to the direction of the applied force. Measurements are carried out on 10 tablets. Results are expressed in terms of the mean, minimum and maximum values (in Newtons) of the force needed to crush the tablets.

Tablets should have a hardness to ensure that they are mechanically robust to withstand packaging and transportation. Furthermore, the tablets should be sufficiently porous to permit ingress of aqueous media to the core.

The cores may likewise be formed using a conventional rotary tablet machine. Cores are preferably compressed under compression forces sufficient to provide cores having a hardness allowing further processing. The cores having the respective hardness must show desired release characteristics. If desired, the cores can be formed at the same time as the tablets are produced. In such case, one might employ a Manesty Dry Cota. Such a press consists of two side-by-side and inter-connected presses where the core is made on one press before being mechanically transferred to the other press for compression coating. Such equipment and techniques for making tablets using such equipment is known in the art and no more needs to be said about this here.

Core and shell material could be manufactured using any conventional manufacturing process for a pharmaceutical dosage form, e.g. direct compression, wet granulation, dry granulation, melt extrusion. Core and shell materials are preferably formed according to wet or dry granulation techniques generally known in the art but not limited to this. In a typical wet granulation procedure, core materials are sieved and blended. Granulating fluid, typically water is then added to the blend and the mixture is homogenized to form a granulate, which is then sprayed dried or dried on a fluid bed drier to obtain a granulate with requisite residual moisture. Preferably the residual moisture content is of from 0.4 to 2.0% by weight but not limited to that. The granulate is then sized by passing it through screens of desired aperture. At this stage, any adjuvants are sized and added to the granulate to form the core composition suitable for compression. The skilled person will appreciate that a shell composition can be formed in an analogous manner.

The resulting formulations in accordance with the present invention show the following advantages:
Formulations approaching, preferably reaching, bioequivalence are achieved;
A relatively high drug loading may easily be achieved;
The formulation of pharmaceutical oral fixed dose combinations with sufficient hardness, resistance to friability, disintegration time etc. is possible;
The sticking tendency and poor flow of the drug substance is reduced to a minimum;
A robust manufacturing process is achieved;
Scale-up of formulation and process resulting in a reproducible performance is achieved; and
Sufficient stability to achieve a reasonable shelf life is achieved.

The invention likewise relates to a process for the preparation of pharmaceutical oral fixed dose combinations as described herein above. Such pharmaceutical oral fixed dose combination may be produced by working up components as defined herein above in the appropriate amounts, to form unit pharmaceutical oral fixed dose combinations.

The pharmaceutical oral fixed dose combinations of the present invention are useful for lowering the blood pressure, either systolic or diastolic or both. The conditions for which the instant invention is useful include, without limitation, hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction (such as Alzheimer's) and stroke, headache and chronic heart failure.

The present invention likewise relates to a method of treating hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure comprising administering to an animal, including human patient, in need of such treatment a therapeutically effective pharmaceutical oral fixed dose combination according to the present invention.

The present invention likewise relates to the use of a pharmaceutical oral fixed dose combination according to the present invention for the manufacture of a medicament for the treatment of hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure.

The present invention likewise relates to a pharmaceutical composition for the treatment of hypertension (whether of the malignant, essential, reno-vascular, diabetic, isolated systolic, or other secondary type), congestive heart failure, angina (whether stable or unstable), myocardial infarction, artherosclerosis, diabetic nephropathy, diabetic cardiac myopathy, renal insufficiency, peripheral vascular disease, left ventricular hypertrophy, cognitive dysfunction, e.g., Alzheimer's, stroke, headache and chronic heart failure, comprising a pharmaceutical oral fixed dose combination according to the present invention.

Ultimately, the exact dose of the active agent and the particular formulation to be administered depend on a number of factors, e.g., the condition to be treated, the desired duration of the treatment and the rate of release of the active agent. For example, the amount of the active agent required and the release rate thereof may be determined on the basis of known in vitro or in vivo techniques, determining how long a particular active agent concentration in the blood plasma remains at an acceptable level for a therapeutic effect.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way.

### EXAMPLE 1

### Bilayer Tablet Formulations

The components of the Aliskiren layer were mixed, granulated and optionally compressed as described herein for preparing a wet-granulated, a roller-compacted or a melt-extrusion-granulated Aliskiren layer, respectively.

The components of the Valsartan layer were mixed, granulated and compressed as described herein. The Valsartan layer was filled into an eccentric tablet press for all bilayer variants and compressed with a compression force of <2.5 kN. The Aliskiren layer was added on top of the Valsartan layer and then the tablet core was compressed with a compaction force of from 5 to 40 kN to obtain a bilayer tablet core.

### EXAMPLE 1.1

### Bilayer Formulations with a Roller Compacted Aliskiren Layer

| | VARIANT 1 | | VARIANT 2 | | VARIANT 3 | |
|---|---|---|---|---|---|---|
| Aliskiren/Valsartan | mg per | % tablet | mg per | % tablet | mg per | % tablet |
| 150/160 mg | unit | weight | unit | weight | unit | weight |
| Aliskiren layer | | | | | | |
| Aliskiren compacted | 338.25 | 47.86 | 338.25 | 47.86 | 338.25 | 47.86 |
| granulate | | | | | | |
| Aliskiren hemifumarate | 165.75 | 23.45 | 165.75 | 23.45 | 165.75 | 23.45 |
| Cellulose MK GR | 159.50 | 22.57 | 152.75 | 21.61 | 146.00 | 20.66 |
| PVP 90F | 6.75 | 0.96 | 13.50 | 1.91 | — | — |
| PVPK30 | — | 0.00 | — | 0.00 | 20.25 | 2.87 |
| Aerosil 200 | 1.00 | 0.14 | 1.00 | 0.14 | 1.00 | 0.14 |
| Mg stearate | 5.25 | 0.74 | 5.25 | 0.74 | 5.25 | 0.74 |
| Mg-Stearat | 5.00 | 0.71 | 5.00 | 0.71 | 5.00 | 0.71 |
| Valsartan layer | | | | | | |
| Vasartan compacted | 307.00 | 43.44 | 307.00 | 43.44 | 307.00 | 43.44 |
| Granulate | | | | | | |
| Valsartan | 160.00 | 22.64 | 160.00 | 22.64 | 160.00 | 22.64 |
| Cellulose MK GR | 108.00 | 15.28 | 108.00 | 15.28 | 108.00 | 15.28 |
| PVP XL | 30.00 | 4.24 | 30.00 | 4.24 | 30.00 | 4.24 |
| Aerosil 200 | 3.00 | 0.42 | 3.00 | 0.42 | 3.00 | 0.42 |
| Mg stearate | 6.00 | 0.85 | 6.00 | 0.85 | 6.00 | 0.85 |
| PVP XL | 18.80 | 2.66 | 18.80 | 2.66 | 18.80 | 2.66 |
| Cellulose MG GR | 35.70 | 5.05 | 35.70 | 5.05 | 35.70 | 5.05 |
| Aerosil 200 | 0.50 | 0.07 | 0.50 | 0.07 | 0.50 | 0.07 |
| Mg-Stearate | 1.50 | 0.21 | 1.50 | 0.21 | 1.50 | 0.21 |
| Total | 706.75 | 100.00 | 706.75 | 100.00 | 706.75 | 100.00 |

| | | | |
|---|---|---|---|
| Hardness [N] (mean) | 258 | 252 | 302 |
| Friability 10 St./6.5 g | 0.23 | 0.23 | |
| 500 U.[%] | | | |
| Disintegration time in | 1′00-1′30 | 1′00-1′30 | 1′00-1′30 |
| min/Valsartan layer | | | |
| Disintegration time in | 22′35-23′00 | 24′00-25′00 | 20′30-22′30 |
| min/Aliskiren layer | | | |

| Aliskiren/Valsartan | VARIANT 4 |
|---|---|
| 300/320 mg | mg/unit |
| Valsartan Layer | |
| Valsartan | 320 |
| Avicel PH 101 | — |
| Avicel PH 102 | 183 |
| PVP K30 | |
| Crospovidone | 31 |
| SDS | |
| LHPC | 62 |
| Aerosil 200 | 6 |
| Magnesium Stearate (Internal) | 12 |
| Magnesium Stearate (External) | 6 |
| Valsartan layer weight | 620 |
| Aliskiren Layer | |
| Aliskiren hemifumarate | 331.5 |
| HPC EXF | |
| Avicel PH 102 | 128.0 |
| Crospovidone | 18 |
| PVP K30 | |
| Mannitol DC | 102 |
| Aerosil 200 | 5.7 |
| Indigotin LAKE 12196 (C) | 0.2 |
| Magnesium Stearate (Internal) | 11.7 |
| Magnesium Stearate (External) | 3 |
| Aliskiren layer weight | 600 |
| Total core weight (mg) | 1220 |
| Coating (2.5%) | 30.5 |
| White Opadry | 20.4 |
| Yellow Opadry | 8.08 |
| Red Opadry | 1.85 |
| Black Opadry | 0.17 |
| Coating weight (mg) | 30.5 |
| Total dosage unit weight (mg) | 1250.5 |

### EXAMPLE 1.2

### Bilayer Formulations with a Wet Granulated Aliskiren Layer

| | VARIANT 1 | | VARIANT 2 | |
|---|---|---|---|---|
| Aliskiren/Valsartan | mg per | % tablet | mg per | % tablet |
| 150/160 mg | unit | weight | unit | weight |
| Aliskiren layer | | | | |
| Aliskiren Granulate | 282.2 | 42.01 | 282.2 | 40.80 |
| Aliskiren hemifumarate | 165.75 | 24.68 | 165.75 | 23.96 |
| Cellulose MK GR | 90.25 | 13.44 | 90.25 | 13.05 |
| Kollidon K30 | 12 | 1.79 | 12 | 1.73 |
| PVPP XL | 14.2 | 2.11 | 14.2 | 2.05 |
| Ethanol denat. 5% Isopropan. | — | | — | |
| Aerosil 200 | 1.8 | 0.27 | 1.8 | 0.26 |
| PVPK30 | 19.6 | 2.92 | 39.6 | 5.73 |
| Mg-Stearate | 5.0 | 0.74 | 5.0 | 0.72 |
| Valsartan layer | | | | |
| Vasartan Granulate | 307.0 | 45.70 | 307.0 | 44.38 |
| Valsartan | 160 | 23.82 | 160 | 23.13 |
| Cellulose MK GR | 108 | 16.08 | 108 | 15.61 |
| PVP XL | 30 | 4.47 | 30 | 4.34 |
| Aerosil 200 | 3 | 0.45 | 3 | 0.43 |
| Mg stearat | 6 | 0.89 | 6 | 0.87 |
| PVP XL | 18.8 | 2.80 | 18.8 | 2.72 |
| Cellulose MG GR | 35.3 | 5.26 | 35.3 | 5.10 |
| Aerosil 200 | 0.5 | 0.07 | 0.5 | 0.07 |
| Mg-Stearate | 1.5 | 0.22 | 1.5 | 0.22 |
| Total | 671.7 | 100.00 | 691.7 | 100.00 |

| | | |
|---|---|---|
| Hardness [N] (mean) | 255.8 | 227.8 |
| Friability 10 St./6.5 g | 0 | 0 |
| 500 U.[%] | | |
| Disintegration time in | 1′00-1′30 | 1′30-1′45 |
| min/Valsartan | | |
| Disintegration time in | 17′30-18′00 | 18′45-20′30 |
| min/Aliskiren | | |

### EXAMPLE 1.3

### Bilayer Formulations with a Melt Granulated Aliskiren Layer. Melt Extrudate: Aliskiren and HPC

| Aliskiren/ | | Composition | Composition |
|---|---|---|---|
| Valsartan | | per | per |
| 300/320 mg | | unit [mg/unit] | unit [%] |
| Aliskiren | Aliskiren hemifumarate | 331.50 | 29.59 |
| layer | HPC* | 45.20 | 4.03 |
| | Avicel 102 (MCC) | 68.05 | 6.07 |
| | Crospovidone XL | 50.00 | 4.46 |
| | Aerosil 200 | 2.50 | 0.22 |
| | Indigotin-farBlack | 0.50 | 0.04 |
| | Magnesium stearate | 2.50 | 0.22 |
| Valsartan | Valsartan | 320.00 | 28.57 |
| layer | Avicel 102 (MCC) | 229.50 | 20.49 |
| | Crospovidone XL | 46.50 | 4.15 |
| | Aerosil 200 | 6.00 | 0.54 |
| | Mg stearate (internal) | 12.00 | 1.07 |
| | Mg-Stearate (external) | 6.00 | 0.54 |
| | Total | 1120.25 | 100.00 |

| | |
|---|---|
| Mean Hardness [N] | 260N (220-300N) |
| Friability 10St./6.5 g 500 U [%] | 0.2% |
| Disintegration time for Valsartan layer [minutes] | 6 min-8 min |
| Disintegration time for Aliskiren layer [minutes] | 22-29 min |

HPC*: hydroxypropylcellulose with aqueous viscosity of 300-600 mPas at 10% w/w concentration and 80,000 average molecular weight

### EXAMPLE 1.4

### Bilayer Formulations with a Melt Granulated Aliskiren Layer. Melt Extrudate: Aliskiren and HPC

| Aliskiren/ | | Composition | |
|---|---|---|---|
| Valsartan | | per unit | Composition |
| 150/160 mg | | [mg/unit] | per unit [%] |
| Aliskiren | Aliskiren hemifumarate | 165.75 | 29.60 |
| layer | HPC* | 12.00 | 2.14 |
| | Cellulose MKGR | 44.63 | 7.97 |
| | Crospovidone | 25.00 | 4.46 |
| | Aerosil 200 | 1.25 | 0.22 |
| | Indigotin Lake 12196 | 0.13 | 0.02 |
| | Magnesium stearate | 1.25 | 0.22 |
| Valsartan | Valsartan | 160.00 | 28.57 |
| layer | Cellulose MK GR | 108.00 | 19.29 |
| | Crospovidone | 30.00 | 5.36 |
| | Aerosil 200 | 3.00 | 0.54 |
| | Mg stearate (internal) | 6.00 | 1.07 |
| | Mg stearate (external) | 3.00 | 0.54 |
| | Total | 560.0 | 100.00 |

| | |
|---|---|
| Mean Hardness [N] | 220N (190-250) |
| Friability 10St./6.5 g 500 U [%] | 0.1% |
| Disintegration time for Valsartan layer [minutes] | 3 min-6 min |
| Disintegration time for Aliskiren layer [minutes] | 15 min-19 min |

HPC*: hydroxypropylcellulose with aqueous viscosity of 300-600 mPas at 10% w/w concentration and 80,000 average molecular weight

### EXAMPLE 1.5

### Bilayer Formulations with a Melt Granulated Aliskiren Layer. Melt Extrudate: Aliskiren and HPC

| Aliskiren/ | | Composition | Composition |
|---|---|---|---|
| Valsartan | | per | per |
| 300/320 mg | | unit [mg/unit] | unit [%] |
| Aliskiren | Aliskiren hemifumarate | 331.50 | 29.60 |
| layer | HPC* | 24.00 | 2.14 |
| | Cellulose MKGR | 89.25 | 7.97 |
| | Crospovidone | 50.00 | 4.46 |
| | Aerosil 200 | 2.50 | 0.22 |
| | Indigotin Lake 12196 | 0.25 | 0.02 |
| | Magnesium stearate | 2.50 | 0.22 |
| Valsartan | Valsartan | 320.00 | 28.57 |
| layer | Cellulose MK GR | 216.00 | 19.29 |
| | Crospovidone | 60.00 | 5.36 |
| | Aerosil 200 | 6.00 | 0.54 |
| | Mg stearate (internal) | 12.00 | 1.07 |
| | Mg stearate (external) | 6.00 | 0.54 |
| | Total | 1120.00 | 100.00 |

| | |
|---|---|
| Mean Hardness [N] | 240 (210-280N) |
| Friability 10St./6.5 g 500 U [%] | 0.2% |
| Disintegration time for Valsartan layer [minutes] | 3-6 min |
| Disintegration time for Aliskiren layer [minutes] | 17-22 min |

HPC*: hydroxypropylcellulose with aqueous viscosity of 300-600 mPas at 10% w/w concentration and 80,000 average molecular weight

### EXAMPLE 2

### Overencapsulated Tablet Formulations

The components of the inner phase are wet-granulated and mixed with the external phase ingredients and compressed into tablets as set forth in WO2005/089729 to obtain Aliskiren-containing tablets. One such tablet is filled into a capsule.

The components of the backfill are granulated as described in the specification above and added to the capsule.

### EXAMPLE 2.1

### Overencapsulation of Aliskiren Tablet with a Final Blend of Valsartan as Backfill

| | Per capsule | |
|---|---|---|
| Components | (mg) | % |
| Inner phase | | |
| Aliskiren hemifumarate | 165.75 | 48.8 |
| Cellulose MK GR | 90.25 | 26.5 |
| Polyvinylpyrrolidon K30 PH | 6.00 | 1.8 |
| Polyvinylpolypyrrolidon XL | 14.20 | 4.2 |
| Ethanol denat.5% Isopropanol | | |
| PVP-K30 dissolved in granulation liquid | 6.00 | 1.8 |
| Total | 282.20 | |
| External phase | | |
| PVPP-XL | 34.00 | 10.0 |
| Cellulose MK GR | 17.00 | 5.0 |
| Aerosil 200 | 1.80 | 0.5 |
| Magnesium stearate | 5.00 | 1.5 |
| Total | 340.0 | 100.0 |

Backfill

| Components | mg |
|---|---|
| Valsartan | 160.00 |
| PVPK30 | 25.00 |
| Duponol C | 1.20 |
| Avicel PH101 | 50.20 |
| PVPXL | 26.00 |
| Mg Stearate | 2.60 |
| Total | 265.00 |

### EXAMPLE 3

### Dry Coated Tablet

Aliskiren in core—Valsartan in shell:

The core is manufactured using compaction technique, such as roller compaction or slugging, where the active ingredient is mixed with pharmaceutical excipients such as diluents, binders, flow regulating agents or lubricants. For example, the active ingredient a) is mixed with microcrystalline cellulose, mannitol, crospovidone, colloidal silica and magnesium stearate (Ingredients 1 to 6, Example 3-Table 1). The mixture is then compacted using roller compactor (or slugging) into ribbon (or slugs), which are screened through a suitable sieve (e.g. 1.0 or 1.2 mm) to yield a dry granulation. The granulation is then mixed with further pharmaceutical excipients such as Cutina HR, microcrystalline cellulose, colloidal silica and magnesium stearate. The blend is then compressed into a suitable die (8 mm or 9 mm) to yield the inner core.

For the manufacture of the shell, valsartan granulate, as described herein, is mixed with pharmaceutical excipients such as microcrystalline cellulose and magnesium stearate to yield the blend for the shell.

The dry coating of the tablets is carried out as mentioned above.

### EXAMPLE 3.1

### Core Composition

### Roller Compacted Aliskiren Hemifumarate

| | Ingredient | | Amount (mg) |
|---|---|---|---|
| 1 | Aliskiren hemifumarate | Active ingredient a) | 165.75 |
| 2 | Crospovidone | Disintegrating agent | 7.5 |
| 3 | Microcrystalline Cellulose | Diluent/Binder | 19.25 |
| 4 | Colloidal silica | Flow regulating agent | 4.5 |
| 5 | Mannitol | Diluent | 16.5 |
| 6 | Magnesium stearate | Lubricant | 1.5 |
| | External phase | | |
| 7 | Cutina HR | Lubricant | 7.5 |
| 8 | Microcrystalline Cellulose | Diluent/Binder | 27 |
| 9 | Colloidal silica | Flow regulating agent | 0.9 |
| 10 | Magnesium stearate | | 4.6 |
| | Total | | 255 |

### Shell Composition

### Wet Granulation used in the Shell

| | Ingredient | | Amount (mg) |
|---|---|---|---|
| 1 | Valsartan granulate* | Active ingredient b) | 307 |
| 2 | Microcrystalline Cellulose | Diluent/Binder | 386 |
| 3 | Magnesium stearate | Lubricant | 7 |
| | Total | | 700 |

| Valsartan granulate* | mg |
|---|---|
| Valsartan | 160.00 |
| Cellulose MK GR | 108.00 |
| PVP XL | 30.00 |
| Colloidal silica | 3.00 |
| Mg Stearate | 6.00 |
| Mg Stearate | 3.00 |
| Total | 310.00 |

Aliskiren Shell—Valsartan core:

Active ingredient b) is mixed with pharmaceutical excipients such as diluents, binders, disintegrants, flow regulating agents and lubricants, for example calcium phosphate, microcrystalline cellulose, colloidal silica and magnesium stearate, and is compacted to yield ribbons (or slugs). These are screened through a suitable screen (e.g. 1.0 or 1.2 mm) and then mixed with further excipients such as diluents, surfactants, disintegrants and lubricants to yield the blend for the core. This blend is then compressed into a suitable die (e.g. 8 mm or 9 mm) to yield the inner core. For the shell, Aliskiren wet granulate, as described herein, using microcrystalline cellulose, polyvinylpyrolidone and crospovidone is used. Additional excipients such as microcrystalline cellulose, polyvinylpyrolidone, colloidal silica and magnesium stearate are added to the granulate to yield the final blend of the shell. The dry coated tablets are manufactured as described above.

### EXAMPLE 3.2

### Core composition

### Compacted Valsartan

| | Ingredient | | Amount (mg/unit) |
|---|---|---|---|
| | Internal Phase | | |
| 1 | Valsartan | Active ingredient b) | 160.0 |
| 2 | Calcium phosphate | Diluent | 29.1 |
| 3 | Croscarmellose sodium | Disintegrant | 5.0 |
| 4 | Microcrystalline | Diluent/Binder | 22.9 |
| | Cellulose | | |
| 5 | Colloidal silica | Flow regulating agent | 1.1 |
| 6 | Magnesium stearate | Lubricant | 2.3 |
| | External phase | | |
| 7 | Sodium lauryl sulfate | Surfactant | 1.0 |
| 8 | Croscarmellose sodium | Disintegrant | 6.3 |
| 9 | Microcrystalline | Diluent/Binder | 20.0 |
| | Cellulose | | |
| 10 | Magnesium stearate | Lubricant | 2.3 |
| | Total | | 250.00 |

### Shell Composition

### Aliskiren in the Shell

| | Ingredient | | Amount (mg/unit) |
|---|---|---|---|
| | Internal phase | | |
| 1 | Aliskiren hemifumarate | Active ingredient a) | 165.75 |
| 2 | Microcrystalline | Diluent/Binder | 90.25 |
| | Cellulose | | |
| 3 | PVP K30 PH | Binder | 6.00 |
| 4 | Crospovidone | Disintegrant | 14.2 |
| 5 | PVP K30 dissolved in | Binder | 6.00 |
| | granulation liquid | | |
| | External phase | | |
| 6 | PVP K30 | Binder | 70.00 |
| 7 | Microcrystalline | Diluent/Binder | 330.00 |
| | Cellulose | | |
| 8 | Colloidal silica | Flow regulating agent | 11.20 |
| 9 | Magnesium Stearate | Lubricant | 6.60 |
| | Total | | 700.00 |

### EXAMPLE

### Dissolution Testing

The dissolution property of the formulations in accordance with the present invention were confirmed as follows.

For paddle method at pH 4.5 and 1:

The assembly consists of the following: a covered vessel made of glass or other inert, transparent material; a motor, and a paddle formed from a blade and shaft as the stirring element. The vessel is partially immersed in a suitable water bath of any convenient size or placed in a heating jacket. The water bath or heating jacket permits holding the temperature inside the vessels at 37±0.5° during the test and keeping the bath fluid in constant, smooth motion. No part of the assembly, including the environment in which the assembly is placed, contributes significant motion, agitation, or vibration beyond that due to the smoothly rotating stirring element, Apparatus that permits observation of the specimen and stirring element during the test is has the following dimensions and capacities: the height is 160 mm to 210 mm and its inside diameter is 98 mm to 106 mm. Its sides are flanged at the top. A fitted cover may be used to retard evaporation.

The shaft is positioned so that its axis is not more than 2 mm at any point from the vertical axis of the vessel and rotates smoothly without significant wobble. The vertical center line of the blade passes through the axis of the shaft so that the bottom of the blade is flush with the bottom of the shaft. The distance of 25±2 mm between the blade and the inside bottom of the vessel is maintained during the test. The metallic or suitably inert, rigid blade and shaft comprise a single entity. A suitable two-part detachable design may be used provided the assembly remains firmly engaged during the test. The paddle blade and shaft may be coated with a suitable inert coating. The dosage unit is allowed to sink to the bottom of the vessel before rotation of the blade is started. A small, loose piece of nonreactive material such as not more than a few turns of wire helix may be attached to dosage units that would otherwise float. Other validated sinker devices may be used.

For basket method at pH 6.8:

The assembly consists of the following: a covered vessel made of glass or other inert, transparent material; a motor, a metallic drive shaft; and a cylindrical basket. The vessel is partially immersed in a suitable water bath of any convenient size or placed in a heating jacket. The water bath or heating jacket permits holding the temperature inside the vessels at 37±0.5° during the test and keeping the bath fluid in constant, smooth motion. No part of the assembly, including the environment in which the assembly is placed, contributes significant motion, agitation, or vibration beyond that due to the smoothly rotating stirring element. Apparatus that permits observation of the specimen and stirring element during the test is has the following dimensions and capacities: the height is 160 mm to 210 mm and its inside diameter is 98 mm to 106 mm. Its sides are flanged at the top. A fitted cover may be used to retard evaporation.

The shaft is positioned so that its axis is not more than 2 mm at any point from the vertical axis of the vessel and rotates smoothly without significant wobble. A speed regulating device is used that allows the shaft rotation speed to be selected and maintained at 100 rpm. Shaft and basket components of the stirring element are of stainless steel type 316 or equivalent. The dosage unit is placed in a dry basket at the beginning of each test. The distance between the inside of the bottom of the vessel and the basket is maintained at 25±2 mm during the test.

1 L of the Dissolution Medium* is placed in the vessel of the apparatus, the apparatus is assembled, the Dissolution Medium is equilibrated to 37±0.5°, and the thermometer is removed. 1 dosage form (e.g. tablet or capsule) is placed on the apparatus, taking care to exclude air bubbles from the surface of the dosage-form unit, and immediately the apparatus is operated at a rate of 75±3 rpm or 100±3 rpm depending on the pH. Within the time interval specified (e.g. 10, 20, 30, 45, 60, 90 and 120 min.), or at each of the times stated, a specimen (≧1 ml) is withdrawn from a zone midway between the surface of the Dissolution Medium and the top of the rotating blade, not less than 1 cm from the vessel wall. [NOTE—the aliquots withdrawn for analysis are replaced with equal volumes of fresh Dissolution Mediums at 37° or, where it can be shown that replacement of the medium is not necessary, the volume change is corrected in the calculation. The vessel is kept covered for the duration of the test, and the temperature of the mixture under test at suitable times is verified.]. The specimen is filtered through a suitable filter, e.g. a 0.45 μm PVDF filter (Millipore) and the first mls (2 to 3 ml) of the filtrate are discarded. The analysis is performed by HPLC or UV detection. The test is repeated at least 6 times. with additional dosage form units.

* Dissolution medium for pH 4.5:1 L of a buffered aqueous solution, adjusted to pH 4.5±0.05 (0.1 M Phosphate buffer solution obtained by dissolving 13.61 g of potassium hydrogen phosphate in 750 ml of deionized water and diluted to 1 L with deionized water)

Dissolution medium for pH 1:1 L of 0.1 M hydrogen chloride.

Dissolution medium for pH 6.8:1 L of a buffered aqueous solution, adjusted to pH 6.8±0.05 (0.05 M phosphate buffer solution obtained by dissolving 6.8 g of potassium hydrogen phosphate and 0.9 g sodium hydroxide in 1 L deionized water).

The examples of pharmaceutical oral fixed dose combinations of the present invention prepared according to the present invention all had the required dissolution characteristics as set forth in the claims of the present invention. The results are shown in the table below.

Table for dissolution data:

| | Dissolution profile | Dissolution profile | Dissolution profile | Dissolution profile |
|---|---|---|---|---|
| | of Aliskiren at pH | of Aliskiren at pH | of Valsartan at pH | of Valsartan at pH |
| | 4.5 after 10 min | 4.5 after 20 min | 4.5 after 30 min | 4.5 after 60 min |
| Example 1.1 | 39 | 70 | 76 | 92 |
| Variant 1 | | | | |
| Example 1.1 | 37 | 67 | 77 | 93 |
| Variant 2 | | | | |
| Example 1.1 | 50 | 82 | 88 | 100 |
| Variant 3 | | | | |
| Example 1.1 | 45 | 82 | 54 | 74 |
| Variant 4 | | | | |
| Example 1.2 | 47 | 81 | 76 | |
| Variant 1 | | | | |
| Example 1.2 | 40 | 71 | 77 | 94 |
| Variant 2 | | | | |
| Example 1.3 | 22 | 46 | 44 | 63 |
| Example 1.4 | 31 | 65 | 55 | 78 (45 min) |
| Example 1.5 | 27 | 59 | 50 | 58 (45 min) |
| Example 2.1 | 32 | 69.7 | 68.3 | 82.1 |
| Example 3.1 | 40 | 72 | 94.3 | 102.8 |
| Example 3.2 | 76 | 96 | 98.22 | 99.5 |

### EXAMPLE

### Bioequivalence Testing

The bioavailability of the pharmaceutical oral fixed dose combinations of the present invention was compared with that of the corresponding free dose combinations. The test (fixed dose combination) and the reference (free dose combination) dosage forms were administered orally to the subjects, and plasma samples were collected over a 48-hour time period. The plasma samples were analyzed for concentration of Valsartan and Aliskiren. Statistical comparison was performed on the maximum plasma concentration (Cmax) achieved with the test and reference and on the area under the plasma concentration vs. time curve (AUC).

The examples of pharmaceutical oral fixed dose combinations of the present invention of Valsartan and Aliskiren (160/150 mg) made in accordance with the present invention was compared with a free dose combination of 160 mg Valsartan and 150 mg Aliskiren tablets in an open-label, randomized, single dose, three period, crossover study in healthy human volunteers. The bioavailability of the fixed dose combination tablets of Valsartan and Aliskiren were compared with the free dose combination, and the 90% confidence interval for AUC and Cmax ratios were within the interval of 0.80-1.25 for Aliskiren and Valsartan, respectively. The results are shown in the table below.

Table for bioequivalence data:

| | Aliskiren | | | | Valsartan | | | |
|---|---|---|---|---|---|---|---|---|
| | mean | % CI | Aliskiren | % CI | mean | % CI | Valsartan | % CI |
| Type of BE | AUC ratio | (90%) | Mean | (90%) | AUC ratio | (90%) | Mean | (90%) |
| study | (AUCinf) | AUC | Cmax ratio | Cmax | (AUCinf) | AUC | Cmax ratio | Cmax |
| Example 1.1 | 0.90 | 0.79-1.02 | 0.71 | 0.58-0.86 | 1.07 | 0.99-1.17 | 1.00 | 0.88-1.13 |
| Variant 4 | | | | | | | | |
| n = 42 subjects | | | | | | | | |
| Example 1.3 | 0.94 | 0.83-1.07 | 0.72 | 0.60-0.88 | 1.01 | 0.91-1.12 | 0.91 | 0.77-1.08 |
| n = 42 subjects | | | | | | | | |
| Example 1.4 | 0.90 | 0.81-1.01 | 0.74 | 0.62-0.89 | 1.03 | 0.92-1.15 | 0.97 | 0.84-1.12 |
| n = 36 | | | | | | | | |
| Example 1.5 | 1.00 | 0.92-1.09 | 0.97 | 0.85-1.10 | 1.11 | 1.02-1.19 | 1.09 | 0.98-1.20 |
| n = 78 subjects | | | | | | | | |
| Example 2.1 | 1.05 | 0.97-1.14 | 1.02 | 0.90-1.15 | 1.04 | 0.98-1.11 | 1.03 | 0.94-1.14 |
| n = 85 subjects | | | | | | | | |

Abbreviations:

Cmax=maximum (peak) observed plasma drug concentration after single dose administration (ng/mL)

AUC=area under the plasma concentration time curve

CI=confidence interval

AUCO-∞=AUC inf=AUC from time zero to infinity (ng.hr/mL)

AUCO-tlast=AUC from time zero to the last measurable concentration sampling time (tlast) (ng.hr/mL)

1. A pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
wherein the pharmaceutical oral fixed dose combination shows an in vitro dissolution of component a) of 80% or less after 10 minutes and 98% or less after 20 minutes, and a dissolution profile of component b) of 25% or more after 30 minutes, and 40% or more after 60 minutes at pH 4.5.

2. A pharmaceutical oral fixed dose combination according to claim 1, wherein the pharmaceutical oral fixed dose combination shows an in vitro dissolution of component a) of 60% or less after 10 minutes and 95% or less after 20 minutes, and a dissolution profile of component b) of 25% or more after 30 minutes, and 45% or more after 60 minutes at pH 4.5.

3. A pharmaceutical oral fixed dose combination according to claim 1, wherein the pharmaceutical oral fixed dose combination shows an in vitro dissolution of component a) of from 60% to 15%, after 10 minutes and of from 95% to 40%, after 20 minutes, and a dissolution profile of component b) of 30% or more, after 30 minutes, and 40% or more after 60 minutes at pH 4.5.

4. A pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
wherein the pharmaceutical oral fixed dose combination shows an in vitro dissolution of component a) of 60% or less after 10 minutes and 95% or less after 20 minutes, and a dissolution profile of component b) of 40% or less after 30 minutes, and 50% or less after 60 minutes at pH 1.

5. A pharmaceutical oral fixed dose combination comprising
a) a therapeutically effective amount of Aliskiren, or a pharmaceutically acceptable salt thereof,
b) a therapeutically effective amount of Valsartan, or a pharmaceutically acceptable salt thereof,
wherein the pharmaceutical oral fixed dose combination shows an in vitro dissolution of component a) of 50% or less after 10 minutes and 95% or less, preferably of from 95% to 30% after 20 minutes, and a dissolution profile of component b) of 75% or more after 30 minutes, and 85% or more after 60 minutes at pH 6.8.

6. The pharmaceutical oral fixed dose combination according claim 1, having an asynchronous release profile of component a) and component b).

7. The pharmaceutical oral fixed dose combination according to claim 1, having a continuous release of both components a) and b).

8. The pharmaceutical oral fixed dose combination according to claim 1, wherein the release of component a) is modified by delaying the time of release or by slowing down the release rate.

9. The pharmaceutical oral fixed dose combination according to claim 1, wherein component b) exhibits immediate release.

10. The pharmaceutical oral fixed dose combination according to claim 1, wherein the pharmaceutical oral fixed dose combination is a solid dosage form.

11. The pharmaceutical oral fixed dose combination according to claim 1, wherein component a) is physically separated from component b).

12. The pharmaceutical oral fixed dose combination according to claim 1, in the form of a multilayer tablet, preferably a bilayer, comprising a layer comprising component a), and a layer comprising component b).

13. The pharmaceutical oral fixed dose combination according to claim 12 wherein the layer containing component a) is obtainable by roller compaction.

14. The pharmaceutical oral fixed dose combination according to claim 13 further comprising a binder in the layer comprising component a) in an amount of 0.7 to 5.0% by weight of the multilayer tablet (prior to any optional film coating).

15. The pharmaceutical oral fixed dose combination according to claim 13 wherein the layer comprising component a) does not include a disintegrant.

16. The pharmaceutical oral fixed dose combination according to claim 12 wherein the layer containing component a) is obtainable by wet granulation.

17. The pharmaceutical oral fixed dose combination according to claim 16 further comprising a binder in the layer comprising component a) in an amount of 2 to 10% by weight of the bilayer tablet (prior to any optional film coating).

18. The pharmaceutical oral fixed dose combination according to claim 12 wherein the layer containing component a) is obtainable by melt extrusion.

19. The pharmaceutical oral fixed dose combination according to claim 1, in the form of an overencapsulated tablet comprising a tablet comprising component a), and multiparticulates comprising component b) both filled into a capsule.

20. The pharmaceutical oral fixed dose combination according to claim 1, in the form of a molded delivery system comprising two matrix zones, one containing component a) and the other containing component b).

21. The pharmaceutical oral fixed dose combination according to claim 20, further comprising an erodible coating layer having at least one opening exposing at least one surface of each of the two matrix zones.

22. The pharmaceutical oral fixed dose combination according to claim 1 comprising a core comprising component a), said core being surrounded by a shell comprising component b).

23. The pharmaceutical oral fixed dose combination according to claim 1 comprising a core comprising component b), said core being surrounded by a shell comprising component a).

24. The pharmaceutical oral fixed dose combination according to claim 1, wherein component a) is present in an amount ranging from about 75 to about 300 mg of the free base per unit dosage form.

25. The pharmaceutical oral fixed dose combination according to claim 1, wherein component b) is present in an amount ranging from about 80 to about 320 mg per unit dosage form.

26. A method of treating hypertension, congestive heart failure, angina, myocardial infarction, artherosclerosis, diabetic nephropathy, diabetic cardiac myopathy, renal insufficiency, peripheral vascular disease, left ventricular hypertrophy, cognitive dysfunction, stroke, headache and chronic heart failure, comprising administering a therapeutically effective amount of the pharmaceutical oral fixed dose combination of claim 1 to a mammal in need thereof.

27. A method for the preparation of a pharmaceutical oral fixed dose combination according to claim 1, in particular a bilayer tablet, said method comprising the steps of (1) granulating component a) and pharmaceutically acceptable additives, optionally in the presence of a granulation liquid, to form an Aliskiren granulate; (2) granulating component b) and pharmaceutically acceptable additives to form a Valsartan granulate; (3) optionally drying resulting respective granulates; (4) sieving; (5) optionally mixing the respective granulates with outer phase excipients; and (6) compressing the Valsartan granulates and the Aliskiren granulates together to form a bilayer tablet.

28. A method for the preparation of a pharmaceutical oral fixed dose combination according to claim 1, said method comprising the steps of (1) granulating component a) and pharmaceutically acceptable additives, optionally in the presence of a granulation liquid, to form an Aliskiren granulate; (2) granulating component b) and pharmaceutically acceptable additives to form a Valsartan granulate; (3) optionally drying resulting respective granulates; (4) sieving; (5) optionally mixing the respective granulates with outer phase excipients; and (6) compressing Aliskiren granulates to form a tablet (s) which is overencapsulated with valsartan granulate.

29. The pharmaceutical oral fixed dose combination according to claim 1, wherein component a) is present in an amount of from about 15 to about 35% by weight based on the total weight of the oral dosage form.

30. The pharmaceutical oral fixed dose combination according to claim 1, wherein component a) is present in an amount of about 20% or more by weight based on the total weight of the oral dosage form.

31. The pharmaceutical oral fixed dose combination according to claim 1, in the form of a multilayer tablet, wherein component a) is present in an amount of from about 40% to about 70%, by weight based on the total weight of the layer comprising component a).

32. The pharmaceutical oral fixed dose combination according to claim 1, in the form of a multilayer tablet, wherein component a) is present in an amount of 60% or more by weight based on the total weight of the layer comprising component a).

33. The pharmaceutical oral fixed dose combination according to claim 1, wherein component a) is present in an amount of from 70% to 95% by weight based on the total weight of the granules comprising component a).

34. The pharmaceutical oral fixed dose combination according to claim 1, wherein component a) is present in an amount of 84% or more by weight based on the total weight of the granules comprising component a).

35. The pharmaceutical oral fixed dose combination according to claim 1, wherein component b) is present in an amount of from 15 to 40% by weight based on the total weight of the oral dosage form.

36. The pharmaceutical oral fixed dose combination according to claim 1, wherein component b) is present in an amount of 20% or more, by weight based on the total weight of the oral dosage form.

37. The pharmaceutical oral fixed dose combination according to claim 1, in the form of a multilayer tablet, wherein component b) is present in an amount of from 30% to 70% by weight based on the total weight of the layer comprising component b).

38. The pharmaceutical oral fixed dose combination according to claim 1, in the form of a multilayer tablet, wherein component b) is present in an amount of 50% or more by weight based on the total weight of the layer comprising component b).

39. The pharmaceutical and fixed dose combination according to claim 12, wherein the multilayer tablet is a bilayer tablet.

40. The method according to claim 26, wherein the fixed dose combination treats hypertension and wherein the mammal is a human.
